(19) United States
(12) Patent Application Publication
(10) Pub. No.: US 2024/0207553 A1
(43) Pub. Date: Jun. 27, 2024

(54) NASAL MINUTE VENTILATION AND PEAK INSPIRATORY FLOW IN RESPIRATORY FLOW THERAPY SYSTEMS

(71) Applicant: Fisher & Paykel Healthcare Limited, Auckland (NZ)

(72) Inventor: Anton Kim Gulley, Auckland (NZ)

(21) Appl. No.: 18/263,994

(22) PCT Filed: Feb. 3, 2021
(86) PCT No.: PCT/IB2022/050932

§ 371 Date: Aug. 2, 2023

Related U.S. Application Data
(60) Provisional application No. 63/145,342, filed on Feb. 3, 2021.

(51) Int. Cl. A61M 16/00; A61M 16/06; A61M 16/10

(52) U.S. Cl. CPC A61M 16/026; A61M 16/0003; A61M 16/0051; A61M 16/0672; A61M 16/109; A61M 2016/0033; A61M 2205/18; A61M 2205/3303; A61M 2205/3327; A61M 2205/3334; A61M 2205/505; A61M 2210/0618; A61M 2230/40

(57) ABSTRACT

Systems and methods determining a nasal minute ventilation and peak inspiratory flow in unsealed respiratory therapy systems. The device can receive data of a parameter of a flow of gases of the respiratory device indicating the patient's respiration. The controller can process the data of the parameter of the flow of gases to remove noise. The controller can determine a device minute ventilation and convert the device minute ventilation to a nasal minute ventilation. The controller can monitor and/or display data relating to at least one of the nasal minute ventilation, nasal minute ventilation rate of change, and nasal minute ventilation trends. The respiratory device can trigger an alarm or notification when at least one of the nasal minute ventilation, the nasal minute ventilation rate of change, and the nasal minute ventilation trends exceeds or falls below a threshold.

### FIELD OF THE DISCLOSURE

The present disclosure relates to methods and systems for providing a respiratory flow therapy to a patient. In particular, the present disclosure relates to estimating one or more respiratory parameters in an unsealed respiratory flow therapy system (i.e., in an open respiratory therapy system that uses an unsealed patient interface).

### BACKGROUND

Breathing assistance apparatuses are used in various environments such as hospital, medical facility, residential care, or home environments to deliver a flow of gases to users or patients. A breathing assistance or respiratory therapy apparatus (collectively, “respiratory apparatus” or “respiratory devices”) may be used to deliver supplementary oxygen or other gases with a flow of gases, and/or a humidification apparatus to deliver heated and humidified gases. A respiratory apparatus may allow adjustment and control over characteristics of the gases flow, including flow rate, temperature, gases concentration, humidity, pressure, etc. Sensors, such as flow sensors and/or pressure sensors are used to measure characteristics of the gases flow.

### SUMMARY

Respiratory devices can monitor and determine various parameters related to a patient's use of the device (i.e. known as respiratory parameters). The parameter data (i.e. respiratory parameters) can inform clinicians about a patient's health, use of the respiratory devices and/or progress in the patient's respiratory functions. The data can assist in determining effectiveness of high flow therapy provided to the patient. The data can also be used to improve the functionality of the respiratory device itself.

Inspiration and expiration by a patient using a respiratory device can affect the gases flow in the device. This is because when the patient inhales through a patient interface, such as a mask or nasal cannula, the resistance to the gases flow in the patient interface decreases; when the patient exhales, the resistance to the gases flow in the patient interface increases. Some parameters, such as respiratory rate, are determined by monitoring variations due to the inspiration and expiration in a flow parameter signal.

In a sealed system, this inhalation and exhalation is relatively easy to measure, due to the use of a sealed interface. However, in an unsealed system (i.e., open system that uses an unsealed interface), such as a nasal high flow system that uses an unsealed patient interface, patient inhalation and exhalation is more difficult to determine because of the open nature (i.e. unsealed nature) of the system. It can be easy to mistake an irregularity in a signal, in particular, a time domain signal, as a respiratory triggering event. The parameter determined from such analyses can be misleading when the respiratory device may detect a breath in the signal when there is no breath (for example, due to the patient being detached, not breathing through the nose, and/or other reasons).

The present disclosure relates to better understanding a patient's breathing, in particular determining respiratory parameters of a patient in an unsealed high flow system. This can be achieved by performing an analysis of gas flow parameters to estimate breathing parameters (i.e. respiratory parameters). The estimated breathing parameters (i.e. respiratory parameters) can include a nasal minute ventilation (i.e. patient minute ventilation) and/or a patient peak inspiratory flow. These breathing parameters (i.e. respiratory parameters) can be used to visually present trend data (e.g., a time series plot over a period of time). The trend data can be used to provide alarms, prompts and/or notifications to a user or clinician to adjust the therapy parameters (e.g., flow rate) to improve therapy outcomes.

In a configuration, a respiratory device is configured to deliver a respiratory therapy to a patient using an unsealed respiratory interface (i.e., unsealed patient interface), the device configured to provide information related to the patient's breathing, the device comprising: a controller, wherein the controller is configured to: receive data of a parameter of a flow of gases of the respiratory device while the device is in use with an unsealed user interface, the parameter indicative of the patient's respiration, determine a device minute ventilation or a parameter indicative of device minute ventilation, and provide an indication of minute ventilation to a user (i.e. minute ventilation of a user). The indication of minute ventilation provided to the patient (i.e. minute ventilation of the patient) is indicative of the amount of respiratory gases that is provided to the user per minute. The minute ventilation is nasal minute ventilation (i.e. patient minute ventilation) when an unsealed nasal interface is used such as for example a nasal cannula.

In a configuration, the controller is further configured to process the data of the parameter of the flow of gases to remove noise. In a configuration, the controller is configured to remove noise relating to the effect of a motor on the parameter of the flow of gases. In a configuration, the controller is configured to receive data regarding a motor speed, and the parameter of the flow of gases is discarded if the motor speed is below a pre-set threshold. In a configuration, the controller is configured to discard the parameter of the flow of gases if the controller determines the parameter of the flow of gases is of insufficient quality. In a configuration, the parameter of the flow of gases is of insufficient quality because it includes large transient peaks.

In a configuration, determining a device minute ventilation comprises utilizing a mathematical function to the parameter of a flow of gases. The function may comprise fitting one or more curves. In one example the device minute ventilation comprises fitting a plurality of splines to the data of the parameter of the flow of gases, wherein the plurality of splines are fit using the least squares criterion and the device minute ventilation is determined by integrating along the plurality of splines. The function is used to average the measured data and determine a best fit. In a configuration, determining a device minute ventilation comprises the integral of the absolute value of the first term of a line fitted to the data of the parameter of the flow of gases. In a configuration, determining a device minute ventilation comprises the integral of the absolute value of a line fitted to the data of the parameter of the flow of gases, divided by a time range. In a configuration, determining a device minute ventilation comprises average of an absolute values of a line fitted to the data of the parameter of the flow of gases across a range of time-points within a time range.

In a configuration, the device minute ventilation is converted to a nasal minute ventilation (i.e., patient minute ventilation) to indicate the amount of gases provided to the user in a minute. In a configuration, the device minute ventilation is converted to a nasal minute ventilation (i.e., patient minute ventilation) using a scalar calibration constant. In a configuration, the scalar calibration constant is determined by inputting patient interface parameters related to the nasal cannula or current flow rate. In a configuration, the scalar calibration constant is determined by inputting at least one of the cannula type, patient size, and naris diameter and/or amount of occlusion of the nares of the user. In a configuration, the scalar calibration constant is calculated by temporarily placing a sealed face mask over a patient's face while the patient is wearing an unsealed nasal cannula to measure at least one flow parameter of the respiratory device.

In a configuration, the controller is further configured to monitor at least one of the nasal minute ventilation, nasal minute ventilation rate of change, and nasal minute ventilation trends. In some examples, these parameters are patient parameters.

In a configuration, the respiratory device further comprises a display, wherein the display is configured to display data relating to the nasal minute ventilation. In a configuration, the respiratory device further comprises a display, wherein the display is configured to display data relating to the nasal minute ventilation rate of change. In a configuration, the respiratory device further comprises a display, wherein the display is configured to display data relating to the nasal minute ventilation trends. The nasal minute ventilation value (i.e., number) may be displayed on the display of the device. The respiratory device may also comprise one or more wireless communication modules e.g. a cellular communication module and/or WIFI module and/or short range communication modules such as Bluetooth. The determined nasal minute ventilation may be transmitted by the respiratory device to a remote server via the communications module at regular intervals (e.g., after a therapy session or at regular intervals during therapy). The server may store or process the nasal minute ventilation. The server may be configured to include the nasal minute ventilation information in an appropriate therapy report for a particular patient. The server is configured to communicate with multiple therapy devices and receive nasal minute ventilation data for multiple patients and manage this data to generate reports etc.

In a configuration, the respiratory device is configured to trigger an alarm or notification when the nasal minute ventilation exceeds or falls below a preset threshold. In a configuration, the respiratory device is configured to trigger an alarm or notification when the nasal minute ventilation rate of change exceeds or falls below a preset threshold. In a configuration, the respiratory device is configured to trigger an alarm or notification when the nasal minute ventilation trends exceeds or falls below a preset threshold.

In a configuration, the respiratory device comprises a patient interface, wherein the patient interface comprises a nasal cannula.

In a configuration, the respiratory device is configured to deliver a nasal high flow therapy.

In a configuration, the respiratory device further comprising a humidifier configured to humidify the gases flow to the patient.

In a configuration, a respiratory device is configured to deliver a respiratory therapy to a patient using an unsealed respiratory interface, the device configured to provide information related to the patient's breathing, the device comprising: a controller, wherein the controller is configured to: receive data of a parameter of a flow of gases of the respiratory device, the parameter indicative of the patient's respiration, process the data of the parameter of the flow of gases to remove noise, determine whether the parameter of the flow of gases is of sufficient quality, determine a device minute ventilation, and convert the device minute ventilation to a nasal minute ventilation using a scalar calibration constant.

In a configuration, the controller is configured to remove noise relating to the effect of a motor on the parameter of the flow of gases. In a configuration, the controller is configured to receive data regarding a motor speed, and the parameter of the flow of gases is discarded if the motor speed is below a pre-set threshold. In a configuration, the parameter of the flow of gases is of insufficient quality because it includes large transient peaks.

In a configuration, wherein determining a device minute ventilation comprises fitting a plurality of splines to the data of the parameter of the flow of gases, wherein the plurality of splines are fit using the least squares criterion and the device minute ventilation is determined by integrating along the plurality of splines. In a configuration, wherein determining a device minute ventilation comprises the integral of the absolute value of the first term of a line fitted to the data of the parameter of the flow of gases. In a configuration, wherein determining a device minute ventilation comprises the integral of the absolute value of a line fitted to the data of the parameter of the flow of gases, divided by a time range. In a configuration, wherein determining a device minute ventilation comprises an average of absolute values of a line fitted to the data of the parameter of the flow of gases across a range of time-points within a time range across a range of time-points within a time range.

In a configuration, the controller is configured to compute a normalized device minute ventilation based on the device minute ventilation. In a configuration, the controller is configured to calculate a noise correction factor correlated to the normalized device minute ventilation. In a configuration, the controller is configured to calculate a corrected device minute ventilation by relating the normalized device minute ventilation and the noise correction factor with the device minute ventilation

In a configuration, wherein the scalar calibration constant is determined by inputting patient interface parameters related to the nasal cannula or current flow rate. In a configuration, wherein the scalar calibration constant is determined by inputting at least one of the cannula type, patient size, and naris diameter. In a configuration, wherein the scalar calibration constant is calculated by temporarily placing a sealed face mask over a patient's face while the patient is wearing a nasal cannula to measure at least one flow parameter of the respiratory device.

In a configuration, wherein the controller is further configured to monitor at least one of the nasal minute ventilation, nasal minute ventilation rate of change, and nasal minute ventilation trends. These parameters may be transmitted to a remote server via the communications module.

In a configuration, wherein the respiratory device further comprises a display, wherein the display is configured to display data relating to the nasal minute ventilation. In a configuration, wherein the respiratory device further comprises a display, wherein the display is configured to display data relating to the nasal minute ventilation rate of change. In a configuration, wherein the respiratory device further comprises a display, wherein the display is configured to display data relating to the nasal minute ventilation trends.

In a configuration, wherein the respiratory device is configured to trigger an alarm or notification when the nasal minute ventilation exceeds or falls below a preset threshold. In a configuration, wherein the respiratory device is configured to trigger an alarm or notification when the nasal minute ventilation rate of change exceeds or falls below a preset threshold. In a configuration, wherein the respiratory device is configured to trigger an alarm or notification when the nasal minute ventilation trends exceeds or falls below a preset threshold.

In a configuration, wherein the respiratory device comprises a patient interface, wherein the patient interface comprises a nasal cannula.

In a configuration, wherein the respiratory device is configured to deliver a nasal high flow therapy.

In a configuration, the respiratory device comprising a humidifier configured to humidify the gases flow to the patient.

In a configuration, the respiratory device is configured to deliver a respiratory therapy to a patient using an unsealed respiratory interface, the device configured to provide information related to the patient's breathing, the device comprising: a controller, wherein the controller is configured to: receive data of a parameter of a flow of gases of the respiratory device, the parameter indicative of the patient's respiration, and determine a patient peak inspiratory flow.

In a configuration, the controller is further configured to determine a device minute ventilation.

In a configuration, the patient peak inspiratory flow is based on the device minute ventilation.

In a configuration, the controller is further configured to process the data of the parameter of the flow of gases to remove noise. In a configuration, the controller is configured to remove noise relating to the effect of a motor on the parameter of the flow of gases. In a configuration, the controller is configured to receive data regarding a motor speed, and the parameter of the flow of gases is discarded if the motor speed is below a pre-set threshold. In a configuration, the controller is configured to discard the parameter of the flow of gases if the controller determines the parameter of the flow of gases is of insufficient quality. In a configuration, the parameter of the flow of gases is of insufficient quality because it includes large transient peaks.

In a configuration, wherein determining the device minute ventilation comprises fitting a plurality of splines to the data of the parameter of the flow of gases, wherein the plurality of splines are fit using the least squares criterion and the device minute ventilation is determined by integrating along the plurality of splines. In a configuration, wherein determining the device minute ventilation comprises the integral of the absolute value of the first term of a line fitted to the data of the parameter of the flow of gases. In a configuration, wherein determining the device minute ventilation comprises the integral of the absolute value of a line fitted to the data of the parameter of the flow of gases, divided by a time range. In a configuration, wherein determining the device minute ventilation comprises an average of absolute values of a line fitted to the data of the parameter of the flow of gases across a range of time-points within a time range.

In a configuration, the controller is configured to compute a normalized device minute ventilation based on the device minute ventilation. In a configuration, the controller is configured to compute a normalized device minute ventilation based on the device minute ventilation. In a configuration, the controller is configured to calculate a corrected device minute ventilation by relating the normalized device minute ventilation and the noise correction factor with the device minute ventilation.

In a configuration, wherein the controller is further configured to convert the device minute ventilation to a nasal minute ventilation. In a configuration, wherein the device minute ventilation is converted to a nasal minute ventilation using a scalar calibration constant. In a configuration, wherein the scalar calibration constant is determined by inputting patient interface parameters related to the nasal cannula or current flow rate. In a configuration, wherein the scalar calibration constant is determined by inputting at least one of the cannula type, patient size, and naris diameter. In a configuration, wherein the scalar calibration constant is calculated by temporarily placing a sealed face mask over a patient's face while the patient is wearing a nasal cannula to measure at least one flow parameter of the respiratory device. In a configuration, wherein the patient peak inspiratory flow is determined by converting the nasal minute ventilation by applying a calibration constant. In a configuration, wherein the calibration constant is 3. In a configuration, wherein the calibration constant is 4. In a configuration, the calibration constant is 5. In a configuration, the calibration constant is 6. In a configuration, the calibration constant is between 3 and 6.

In a configuration, wherein the controller is further configured to monitor at least the peak inspiratory flow or the rate of change of the peak inspiratory flow.

In a configuration, wherein the respiratory device further comprises a display, wherein the display is configured to display data relating to the peak inspiratory flow. In a configuration, wherein the respiratory device further comprises a display, wherein the display is configured to display data relating to the rate of change of the peak inspiratory flow. In a configuration, wherein the respiratory device is configured to trigger an alarm or notification when the peak inspiratory flow exceeds or falls below a preset threshold.

In a configuration, wherein the respiratory device is configured to trigger an alarm or notification when rate of change of the peak inspiratory flow exceeds or falls below a preset threshold.

In a configuration, wherein the respiratory device comprises a patient interface, wherein the patient interface comprises a nasal cannula.

In a configuration, wherein the respiratory device is configured to deliver a nasal high flow therapy.

In a configuration, the respiratory device further comprising a humidifier configured to humidify the gases flow to the patient.

In a configuration, a respiratory device is configured to deliver a respiratory therapy to a patient using an unsealed respiratory interface, the device configured to provide information related to the patient's breathing, the device comprising: a controller, wherein the controller is configured to: receive a flow rate of a flow of gases of the respiratory device while the device is in use with an unsealed user interface, determine a respiration parameter based on the received flow rate of gases, wherein the respiratory parameter is indicative of patient ventilation.

In a configuration, the controller is configured to display on a graphical user interface the respiration parameter.

In a configuration, the controller is configured to determine a device minute ventilation based on the flow of the gases, and the controller configured to determine the patient ventilation based on the device minute ventilation.

In a configuration, the controller is further configured to process the flow rate of the flow of gases to remove noise.

In a configuration, the controller is configured to remove noise relating to the effect of a motor on the parameter of the flow of gases.

In a configuration, the controller is configured to receive data regarding a motor speed, and the parameter of the flow of gases is discarded if the motor speed is below a pre-set threshold. In a configuration, the controller is configured to discard the flow rate of the flow of gases if the controller determines the flow rate of the flow of gases is of insufficient quality. In a configuration, the flow rate of the flow of gases is of insufficient quality because it includes large transient peaks. In a configuration, the controller is configured to determine the device minute ventilation according to any one of the above configurations, and nasal minute ventilation may be calculated as defined in any of the above configurations. In a configuration, a respiratory device is configured to deliver a respiratory therapy to a patient using an unsealed respiratory interface, the device configured to provide information related to the patient's breathing, the device comprising: a controller, wherein the controller is configured to: receive a flow rate of a flow of gases of the respiratory device while the device is in use with an unsealed user interface, determine a patient peak inspiratory flow based on the received flow rate of gases, wherein the patient peak inspiratory flow is indicative of patient ventilation.

In a configuration, the controller is configured to display the patient peak inspiratory flow on a graphical user interface. In a configuration, the controller is configured to determine the patient peak inspiratory flow based on a device minute ventilation. In a configuration, the device minute ventilation is based on the flow rate of the flow of gases, wherein the flow rate of the flow of gases is pre-processed according to any of the above configurations. In a configuration, the device minute ventilation is calculated according to any of the above configurations. In a configuration, the controller is configured to convert the device minute ventilation to a nasal minute ventilation according to any of the above configurations.

In a configuration, the controller is configured to monitor at least the peak inspiratory flow or the rate of change of the peak inspiratory flow. In a configuration, the respiratory device further comprises a display, the display is configured to display data relating to at least one of a peak inspiratory flow or a rate of change of the peak inspiratory flow. In a configuration, the respiratory is configured to trigger an alarm or notification when the peak inspiratory flow exceeds or falls below a preset threshold. In a configuration, the respiratory is configured to trigger an alarm or notification when the rate of change of the peak inspiratory flow exceeds or falls below a preset threshold.

In a configuration, the controller is configured to determine a tidal volume and trigger an alarm or notification when the tidal volume falls below a preset threshold. In a configuration, the controller is configured to normalize a corrected device minute ventilation using a device flow rate. In a configuration, the respiratory device further comprises a display configured to display data relating to corrected device minute ventilation. In a configuration, the respiratory device further comprises a display configured to display data relating to corrected device minute ventilation rate of change. In a configuration, the respiratory device further comprises a display configured to display data related to corrected device minute ventilation trends. In a configuration, the respiratory device is configured to trigger an alarm or notification when the corrected device minute ventilation exceeds or falls below a present threshold. In a configuration, the respiratory device is configured to trigger an alarm or notification when the corrected device minute ventilation rate of change exceeds or falls below a preset threshold. In a configuration, the respiratory device is configured to trigger an alarm or notification when the corrected device minute ventilation trends exceeds or falls below a preset threshold.

### BRIEF DESCRIPTION OF THE DRAWINGS

These and other features, aspects, and advantages of the present disclosure are described with reference to the drawings of certain embodiments, which are intended to schematically illustrate certain embodiments and not to limit the disclosure.

FIG. 1A illustrates schematically a respiratory system configured to provide a respiratory therapy to a patient.

FIG. 1B illustrates schematically another embodiment of a respiratory system configured to provide a respiratory therapy to a patient.

FIG. 2 illustrates a front view of an embodiment of a respiratory device with a humidification chamber in position and a raised handle/lever.

FIG. 3 illustrates a top view of the embodiment of the respiratory device of FIG. 2.

FIG. 4 illustrates a right side view of the embodiment of the respiratory device of FIG. 2.

FIG. 5 illustrates a left side view of the embodiment of the respiratory device of FIG. 2.

FIG. 6 illustrates a rear view of the embodiment of the respiratory device of FIG. 2.

FIG. 7 illustrates a front left perspective view of the embodiment of the respiratory device of FIG. 2.

FIG. 8 illustrates a front right perspective view of the embodiment of the respiratory device of FIG. 2.

FIG. 9 illustrates a bottom view of the embodiment of the respiratory device of FIG. 2.

FIG. 10 illustrates an embodiment of an air and oxygen inlet arrangement of a respiratory device.

FIG. 11 illustrates another embodiment of an air and oxygen inlet arrangement of the respiratory device.

FIG. 12 illustrates a transverse sectional view showing further detail of the air and oxygen inlet arrangement of FIG. 11.

FIG. 13 illustrates another transverse sectional view showing further detail of the air and oxygen inlet arrangement of FIG. 11.

FIG. 14 illustrates a longitudinal sectional view showing further detail of the air and oxygen inlet arrangement of FIG. 11.

FIG. 15 illustrates an exploded view of upper and lower chassis components of a main housing of the respiratory device.

FIG. 16 illustrates a front left side perspective view of the lower chassis of the main housing showing a housing for receipt of a motor and/or sensor module sub-assembly.

FIG. 17 illustrates a first underside perspective view of the main housing of the respiratory device showing a recess inside the housing for the motor and/or sensor module sub-assembly.

FIG. 18 illustrates a second underside perspective view of the main housing of the respiratory device showing the recess for the motor and/or sensor module sub-assembly.

FIG. 19A illustrates a block diagram of a control system interacting with and/or providing control and direction to components of a respiratory system.

FIG. 19B illustrates a block diagram of an example controller.

FIG. 20 illustrates a block diagram of a motor and sensor module.

FIG. 21 illustrates a sensing chamber of an example motor and sensor module.

FIG. 22A illustrates an embodiment of a flow chart for a method of estimating nasal minute ventilation.

FIG. 22B illustrates another embodiment of a flow chart for a method of estimating nasal minute ventilation.

FIG. 22C illustrates another embodiment of a flow chart for a method of estimating nasal minute ventilation.

FIG. 22D illustrates another embodiment of a flow chart for a method of estimating nasal minute ventilation.

FIG. 23 illustrates another embodiment of a flow chart for a method of estimating device minute ventilation.

FIG. 24 illustrates an embodiment of a flow chat for a method of estimating peak inspiratory flow.

### DETAILED DESCRIPTION

Although certain examples are described below, those of skill in the art will appreciate that the disclosure extends beyond the specifically disclosed examples and/or uses and obvious modifications and equivalents thereof. Thus, it is intended that the scope of the disclosure herein disclosed should not be limited by any particular examples described below.

### Overview of Example Respiratory System

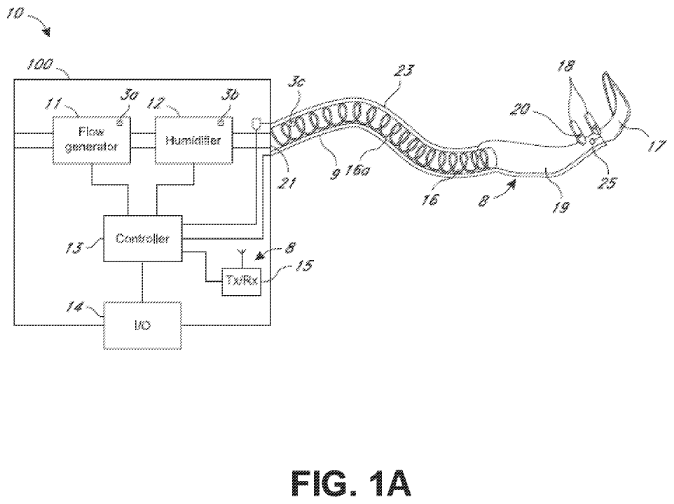

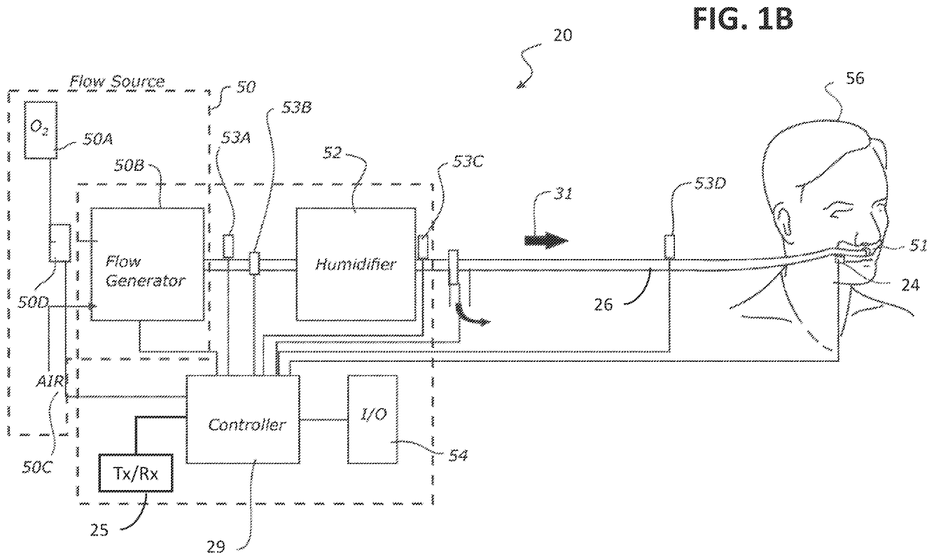

A schematic representation of a respiratory system 10 is provided in FIG. 1A. The respiratory system 10 can include a main device housing 100. The main device housing 100 can contain a flow generator 11 that can be in the form of a motor/impeller arrangement, an optional humidifier or humidification chamber 12, a controller 13, and a user interface 14. The user interface 14 can include a display and input device(s) such as button(s), a touch screen, a combination of a touch screen and button(s), or the like. The controller 13 can include one or more hardware and/or software processors and can be configured or programmed to control the components of the apparatus, including but not limited to operating the flow generator 11 to create a flow of gases for delivery to a patient, operating the humidifier 12 (if present) to humidify and/or heat the gases flow, receiving user input from the user interface 14 for reconfiguration and/or user-defined operation of the respiratory system 10, and outputting information (for example on the display) to the user. The user can be a patient, healthcare professional, or others.

With continued reference to FIG. 1A, a patient breathing conduit 16 can be coupled to a gases flow outlet 21 in the main device housing 100 of the respiratory system 10, and be coupled to a patient interface 17, such as a non-sealing interface like a nasal cannula with a manifold 19 and nasal prongs 18.

The gases flow can be generated by the flow generator 11, and may be humidified, before being delivered to the patient via the patient conduit 16 through the patient interface 17. The controller 13 can control the flow generator 11 to generate a gases flow of a desired flow rate, and/or one or more valves to control mixing of air and oxygen or other breathable gas. The controller 13 can control a heating element in the humidification chamber 12, if present, to heat the gases to a desired temperature that achieves a desired level of temperature and/or humidity for delivery to the patient. The patient conduit 16 can have a heating element 16a, such as a heater wire, to heat gases flow passing through to the patient. The heating element 16a can also be under the control of the controller 13.

The system 10 can use ultrasonic transducer(s), flow sensor(s) such as a thermistor flow sensor, pressure sensor(s), temperature sensor(s), humidity sensor(s), or other sensors, in communication with the controller 13, to monitor characteristics of the gases flow and/or operate the system 10 in a manner that provides suitable therapy. The gases flow characteristics can include gases concentration, flow rate, pressure, temperature, humidity, or others. The sensors 3a, 3b, 3c, 20, 25, such as pressure, temperature, humidity, and/or flow sensors, can be placed in various locations in the main device housing 100, the patient conduit 16, and/or the patient interface 17. The controller 13 can receive output from the sensors to assist it in operating the respiratory system 10 in a manner that provides suitable therapy, such as to determine a suitable target temperature, flow rate, and/or pressure of the gases flow. Providing suitable therapy can include meeting or exceeding a patient's inspiratory demand, so as to reduce entrainment of ambient air at the patient interface.

The system 10 can include a wireless data transmitter and/or receiver, or a transceiver 15 to enable the controller 13 to receive data signals 8 in a wireless manner from the operation sensors and/or to control the various components of the system 10. Additionally, or alternatively, the data transmitter and/or receiver 15 can deliver data to a remote patient management system (i.e. remote server) or enable remote control of the system 10. The system 10 can include a wired connection, for example, using cables or wires, to enable the controller 13 to receive data signals 8 from the operation sensors and/or to control the various components of the system 10. The system can also include one or more wireless communication modules to allow the system to communicate with one or more other devices (e.g., a mobile phone or a remote computing system). The system 10 may include a cellular communication module e.g. a 3G, 4G or 5G module. The system 10 may also include a Bluetooth module and a WiFi module. The system 10 may further include additional wireless communication modules. The wireless communication module allows for two way communication from the respiratory system to a remote patient management system (i.e. remote server). For example, measured nasal minute ventilation data (i.e., patient minute ventilation) may be communicated to a remote server (i.e. a remote patient management system). The remote patient management system may be a single server or a network of servers or a cloud computing system or other suitable architecture for operating a remote patient management system. The remote patient management system (i.e. remote server) further includes memory for storing received data and various software applications or services that are executed to perform multiple functions. Then, for example, the remote patient management system (i.e. remote server) may communicate information or instructions to the system 10 at least in part dependent on the data received. For example, the nature of the data received may trigger the remote server (or a software application running on the remote server) to communicate an alert, alarm, or notification to the system 10. The remote patient management system may further store the received data for access by an authorized party such as a clinician or the patient or another authorized party.

FIG. 1B illustrates another configuration of a respiratory system 20. The schematic representation of the example respiratory system 20 is provided in FIG. 1B. The respiratory system 20 (i.e. “breathing assistance apparatus”) comprises a flow source 50 for providing a high flow gas 31 such as air, oxygen, air blended with oxygen, or a mix of air and/or oxygen and one or more other gases. In some embodiments, the respiratory system 20 can have a connection for coupling to the flow source 50. In some examples, the flow source 50 can form part of the respiratory system 20 or be separate to the respiratory system 20. In some embodiments, a part of the flow source 50 can form a part of the respiratory system 20 and a part of the flow source 50 can form outside of the respiratory system 20. In some examples, the respiratory system 20 can include a combination of components. For example, the respiratory system 20 can be selected from a combination of a flow source, a humidifier for humidifying the gas-flow, an inspiratory tube, a conduit (e.g., dry line or heated breathing tube), a patient interface, a non-return valve, a filter, etc.

In some embodiments, the flow source 50 can include an in-wall supply of oxygen, a tank of oxygen 50A, a tank of other gas and/or a high flow apparatus with a flow generator 50B. FIG. 1B illustrates a flow source 50 with a flow generator 50B. In some embodiments, the flow source 60 can include an inlet 50C and to an O₂ source 50A (such as tank or O2 generator). In some embodiments, the inlet 50C and the O₂ source 50A is connected through a gas flow control 50D. The gas flow control 50D can be, for example, a shut off valve, a regulator, etc. In some examples, the flow generator 50B can control flows delivered to the patient 56 using one or more valve. In some embodiments, the flow generator 50B can comprise a blower. In some examples, the flow source 50 can include a combination of a flow generator 50B, O₂ source 50A, and an air source 50C. As shown in FIG. 1B, the flow source 50 can be part of the apparatus 20. In some embodiments, when the flow source 50 includes an external oxygen tank or in-wall source, the flow source 50 may be considered a separate component, in which case the respiratory system 20 has a connection port to connect to the flow source 50. In some examples, the flow source 50 provides a flow of gas that can be delivered to a patient via a delivery conduit 26, and patient interface 51. As will be discussed in more detail below, the flow source 50 can provide a high flow of gases to the patient.

The respiratory system 20 can include a patient interface 51. In some embodiments, the patient interface 51 can be an unsealed (i.e., non-sealing) interface such as a non-sealing nasal cannula. The unsealed interface can be used, for example, during high flow therapy. In some configurations, the patient interface 51 is a non-sealing patient interface which can be used to help prevent barotrauma (e.g. tissue damage to the lungs or other organs of the respiratory system due to difference in pressure relative to the atmosphere) while high flow therapy is provided to a patient. High flow therapy (i.e., providing high flow rates of heated, humidified gases via sealed interfaces) can cause damage to a patient's lungs and/or airways due to high pressures created in the lungs via the sealed interface. In some configurations, the flow source 50 can provide a base gas flow rate of between, e.g. 0.5 litres/min and 375 litres/min, or any range within that range, or even ranges with higher or lower limits. Details of the ranges and nature of the flow rates will be provided in more detail below.

As shown in FIG. 1B, the respiratory system 20 can include a humidifier 52. In some examples, the humidifier 52 can optionally be provided between the flow source 50 and the patient to provide humidification of the delivered gas. In some configurations the humidifier may be optional, or it may be preferred due to the advantages of humidified gases helping to maintain the condition of the airways.

In some configurations the respiratory system 20 can include one or more sensors 53A, 53B, 53C, 53D can be placed throughout the system and/or at, on, or near the patient 56. In some examples, the sensors 53A, 53B, 53C, 53D are sensors for detecting respiratory system 20 parameters such as flow, oxygen fraction, pressure, humidity, temperature etc. In some configurations, the sensors 53A, 53B, 53C, 53D are sensors for deriving respiratory system 20 parameters such as flow, oxygen fraction, pressure, humidity, temperature etc. In some examples, the sensors 53A, 53B, 53C, 53D can be one or more physiological sensors for sensing patient physiological parameters such as, heart rate, oxygen saturation, partial pressure of oxygen in the blood, respiratory rate, partial pressure of CO2 in the blood. In some configurations, the sensors 53A, 53B, 53C, 53D can be one or more physiological sensors for deriving patient physiological parameters such as, heart rate, oxygen saturation, partial pressure of oxygen in the blood, respiratory rate, partial pressure of CO2 in the blood. In some examples, the respiratory system 20 can include other patient sensors such as EEG sensors, torso bands to detect breathing, and other suitable sensors. In some configurations, one or more of the sensors 53A, 53B, 53C, 53D might form part of the respiratory system 20, or be external thereto, with the respiratory system 20 having inputs for any external sensors. The sensors can be coupled to or send their output to a controller 29.

In some configurations, the respiratory system 20 can include a sensor 24 for measuring the oxygen fraction of air the patient inspires. In some examples, the sensor 24 can be placed on the patient interface 51, to measure or otherwise determine the fraction of oxygen proximate (at/near/close to) the patient's mouth and/or nose. In some configurations, the output from the sensor 24 is sent to a controller 29 to assist control of the respiratory system 20 alter operation accordingly. The controller 29 is coupled to the flow source 50, humidifier 52 and sensor 24. In some configurations, the controller 29 controls these and other aspects of the respiratory system 20 as described herein. In some examples, the controller can operate the flow source 50 to provide the delivered flow of gas at a desired flow rate high enough to meet or exceed a user's (i.e. patient's) inspiratory demand. The flow rate is provided is sufficient that ambient gases are not entrained as the user (i.e. patient) inspires. In some configurations, the sensor 24 can convey measurements of oxygen fraction at the patient mouth and/or nose to a user, who can input the information to the respiratory system 20/controller 29.

In some configurations, the respiratory system 20 can include a breathing conduit 26. In some examples, the breathing conduit 26 can include a non-return valve. In some configurations, a filter or filters may be provided at the air inlet 50C and/or inlets to the flow generator 50B to filter the incoming gases before they are pressurized into a high flow gas 31 to the flow generator 50B.

In some configurations, the respiratory system 20 can be an integrated or a separate component-based arrangement. In some examples, the respiratory system 20 can be a modular arrangement of components. The respiratory system 20 may comprise some or all of the components shown in FIG. 1B. For example, the conduit 26 and patient interface 51 can be separate from the respiratory system 20. The apparatus/system illustrated in FIG. 1B has bene referred to as a breathing assistance apparatus or respiratory system, but this should not be considered limiting. Breathing assistance apparatus and respiratory system will be broadly considered herein to comprise anything that provides a flow rate of gas to a patient. In some configurations, the breathing assistance apparatus and respiratory system can include a detection system that can be used to determine if the flow rate of gas meets inspiratory demand.

In some configurations, the respiratory system 20 can include a main device housing. Although this is not illustrated in FIG. 1B, the main device housing can be similar to the main device housing 100 illustrated in FIG. 1A. In some configurations, the main device housing can contain the flow generator 50B, a controller 29, and an input/output I/O user interface 54. In some configurations, the flow generator 50b can comprise a motor/impeller arrangement. In some examples, the main device housing can also contain the humidifier 52. In some examples, the user interface 54 can include a display and input device(s) such as button(s), a touch screen (e.g. an LCD screen), and a combination of a touch screen and button(s), or the like.

In some configurations, the controller 29 can include one or more hardware and/or software processors and can be configured or programmed to control the components of the system. For example, in some configurations, the controller 29 can be configured to operate the flow generator 50B to create a flow of gases for delivery to a patient, operate the humidifier 52 to humidify and/or heat the gases flow, receive user input from the user interface 54 for reconfiguration and/or user-defined operation of the breathing assistance apparatus 20, and output information to the user (e.g., on the display). The user can be a patient, healthcare professional, or others.

In some examples, as shown in FIG. 1B, the patient breathing conduit 26 can be coupled to a gases flow outlet (gases outlet or patient outlet port) in the main device housing of the respiratory system 20, and be coupled to a patient interface 24, such as a non-sealing interface like a nasal cannula with a manifold and nasal prongs. In some configurations, the patient breathing conduit 26 can also be a tracheostomy interface, or other unsealed interfaces.

In some configurations, the gases flow is generated by the flow generator 50B, and may be humidified, before being delivered to the patient 56 via the patient breathing conduit 26 through the patient interface 51. In some examples, the controller 29 can control the flow generator 50B to generate a gases flow of a desired flow rate, and/or one or more valves to control mixing of air and oxygen or other breathable gas. The controller 29 can control a heating element in or associated with the humidification chamber to heat the gases to a desired temperature. In some configurations, the humidification chamber can help the gases achieve a desired level of temperature and/or humidity for delivery to the patient 56. In some examples, the patient breathing conduit 26 can include a heating element, such as a heater wire, to heat gases flow passing through to the patient 56. An example of the heater wire, though not pictured in FIG. 1B, can be seen in the heating wire 16a of FIG. 1A. In some configurations, the heating element can also be under the control of the controller 29.

In some examples, the humidifier 52 of the respiratory system 20 is configured to combine or introduce humidity with or into the gases flow. The humidifier 52 of the respiratory system 20 can be employed using various configurations. In some configurations, the humidifier 52 can comprise a humidification chamber that is removable. For example, the humidification chamber may be partially or entirely removed or disconnected from the flow path and/or respiratory system 20. The humidification chamber can be removed for refilling, cleaning, replacement and/or repair for example. In some configurations, humidification chamber may be received and retained by or within a humidification compartment or bay of the apparatus, or may otherwise couple onto or within the housing of the apparatus.

In some configurations, the humidification chamber of the humidifier 52 may comprise a gases inlet and a gases outlet to enable connection into the gases flow path of the respiratory system 20. In some examples, the flow of gases from the flow generator 50B is received into the humidification chamber via its gases inlet and exits the chamber via its gases outlet, after being heated and/or humidified. The humidification chamber can contain a volume of liquid, typically water or something similar. In some configurations, the liquid in the humidification chamber is controllably heated by one or more heaters or heating elements associated with the chamber to generate water vapour or steam to increase the humidity of the gases flowing through the chamber. In some examples, the humidifier is a Passover humidifier. In some configurations, the humidifier may be a non-Passover humidifier. In some examples, the humidifier 52 may include a heater plate. The heater plate may, for example, be associated or within a humidification bay that the chamber sits on for heating. In some configurations, the chamber may be provided with a heat transfer surface, (e.g., a metal insert, plate or similar, in the base or other surface of the chamber) that interfaces or engages with the heater plate of the humidifier. In some examples, the humidification chamber may comprise an internal heater or heater elements inside or within the chamber. In some configurations, the internal heater or heater elements may be integrally mounted or provided inside the chamber, or may be removable from the chamber.

In some configurations, the humidification chamber may be any suitable shape and/or size. In some examples, the location, number, size, and/or shape of the gases inlet and gases outlet of the chamber may be varied. In some configurations, the humidification chamber may have a base surface, one or more side walls extending up from the base surface, and an upper or top surface. In some configurations, the gases inlet and gases outlet may be position on the same side of the chamber. In some examples, the gases inlet and gases outlet may be on different surfaces of the chamber, such as on opposite sides or locations, or other different locations. In some configurations, the gases inlet and gases outlet may have parallel flow axes. In some examples, the gases inlet and gases outlet may be positioned at the same height on the chamber.

As discussed above, the system 20 can use ultrasonic transducer(s), flow sensor(s) such as a thermistor flow sensor, pressure sensor(s), temperature sensor(s), humidity sensor(s), or other sensors, in communication with the controller 29, to monitor characteristics of the gases flow and/or operate the system 20 in a manner that provides suitable therapy. The gases flow characteristics can include gases concentration, flow rate, pressure, temperature, humidity, or others. The sensors 53A, 53B, 53C, 53D, 24, such as pressure, temperature, humidity, and/or flow sensors, can be placed in various locations in the main device housing, the patient conduit 26, and/or the patient interface 51. The controller 29 can receive output from the sensors to assist it in operating the breathing assistance apparatus 20 in a manner that provides suitable therapy, such as to determine a suitable target temperature, flow rate, and/or pressure of the gases flow. Providing suitable therapy can include meeting a patient's inspiratory demand. In the illustrated configuration sensors 53A, 53B, and 53C are positioned in the housing of the apparatus, sensor 53D in the patient conduit 26, and sensor 24 in the patient interface 51.

In some configurations, the respiratory system 20 can include one or more communication modules to enable data communication or connection with one or more external devices or servers over a data or communication link or data network, whether wired, wireless, or a combination thereof. In some configurations, the respiratory system 20 can include a wireless data transmitter and/or receiver, or a transceiver 25 to enable the controller 29 to receive data signals in a wireless manner from the operation sensors and/or to control the various components of the respiratory system 20. In some examples, the transceiver 15 or data transmitter and/or receiver module may have an antenna. In some configurations, the transceiver may comprise a Wi-Fi modem. In some examples, the data transmitter and/or receiver 25 can deliver data to a remote server or enable remote control of the respiratory system 20. The respiratory system 20 can include a wired connection, for example, using cables or wires, to enable the controller 29 to receive data signals from the operation sensors and/or to control the various components of the respiratory system 20. The respiratory system 20 can include one or more wireless communication modules. For example, the apparatus may comprise a cellular communication module such as for example a 3G, 4G or 5G module. The module 25 may be or may comprise a modem that enables the apparatus to communicate with a remote server using an appropriate communication network. The communication may be two-way communication between the apparatus and a server or other remote system. The remote system may be a remote patient management system that stores patient data e.g. respiratory rate, nasal minute ventilation (i.e., patient ventilation), measured SpO₂ as well as therapy parameters used e.g. flow rate, humidity, oxygen concentration (i.e., FiO₂). The therapy parameters and patient data may be received for each therapy session and this data can be stored and used to generate one or more reports. The one or more reports are accessible by an authorized party e.g. a clinician or an insurance provider or the patient. The one or more reports may also be transmitted to an authorized party. The respiratory system 20 may also comprise other wireless communication modules such as for example a Bluetooth module and/or a Wi-Fi module. The Bluetooth and/or WiFi module can allow the respiratory system 20 to wirelessly send information to another device such as for example a smartphone or tablet or operate over a LAN (local area network) or Wireless LAN (WLAN). The respiratory system 20 can comprise a Near Field Communication (NFC) module to allow for data transfer and/or data communication.

The respiratory system 10 and/or the respiratory system 20 can comprise a high flow therapy apparatus. High flow therapy as discussed herein is intended to be given its typical ordinary meaning, as understood by a person of skill in the art, which generally refers to a respiratory system delivering a targeted flow of humidified respiratory gases via an intentionally unsealed patient interface with flow rates generally intended to meet or exceed inspiratory flow of a user. Typical patient interfaces include, but are not limited to, a nasal or tracheal patient interface. Typical flow rates for adults often range from, but are not limited to, about fifteen liters per minute to about sixty liters per minute or greater. Typical flow rates for pediatric users (such as neonates, infants and children) often range from, but are not limited to, about one liter per minute per kilogram of user weight to about three liters per minute per kilogram of user weight or greater. High flow therapy can also optionally include gas mixture compositions including supplemental oxygen and/or administration of therapeutic medicaments. High flow therapy is often referred to as nasal high flow (NHF), humidified high flow nasal cannula (HHFNC), high flow nasal oxygen (HFNO), high flow therapy (HFT), or tracheal high flow (THF), among other common names. For example, in some configurations, for an adult patient ‘high flow therapy’ may refer to the delivery of gases to a patient at a flow rate of greater than or equal to about 10 litres per minute (10 LPM), such as between about 10 LPM and about 100 LPM, or between about 15 LPM and about 95 LPM, or between about 20 LPM and about 90 LPM, or between about 25 LPM and about 85 LPM, or between about 30 LPM and about 80 LPM, or between about 35 LPM and about 75 LPM, or between about 40 LPM and about 70 LPM, or between about 45 LPM and about 65 LPM, or between about 50 LPM and about 60 LPM. In some configurations, for a neonatal, infant, or child patient ‘high flow therapy’ may refer to the delivery of gases to a patient at a flow rate of greater than 1 LPM, such as between about 1 LPM and about 25 LPM, or between about 2 LPM and about 25 LPM, or between about 2 LPM and about 5 LPM, or between about 5 LPM and about 25 LPM, or between about 5 LPM and about 10 LPM, or between about 10 LPM and about 25 LPM, or between about 10 LPM and about 20 LPM, or between about 10 LPM and 15 LPM, or between about 20 LPM and 25 LPM. A high flow therapy apparatus with an adult patient, a neonatal, infant, or child patient, may deliver gases to the patient at a flow rate of between about 1 LPM and about 100 LPM, or at a flow rate in any of the sub-ranges outlined above.

High flow therapy can be effective in meeting or exceeding the patient's inspiratory demand, increasing oxygenation of the patient and/or reducing the work of breathing. Additionally, high flow therapy may generate a flushing effect in the nasopharynx such that the anatomical dead space of the upper airways is flushed by the high incoming gases flow. The flushing effect can create a reservoir of fresh gas available of each and every breath, while minimizing re-breathing of carbon dioxide, nitrogen, etc.

The patient interface for use in a high flow therapy can be a non-sealing interface to prevent barotrauma, which can include tissue damage to the lungs or other organs of the patient's respiratory system due to difference in pressure relative to the atmosphere. The patient interface can be a nasal cannula with a manifold and nasal prongs or an unsealed tracheal interface.

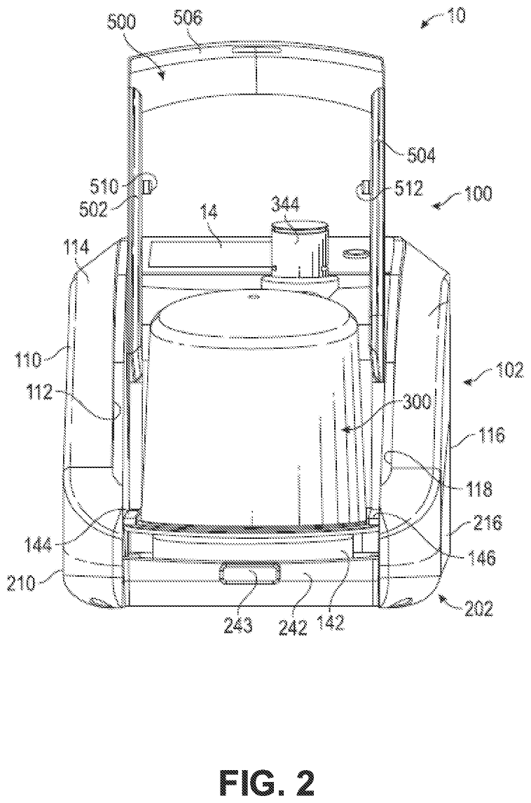

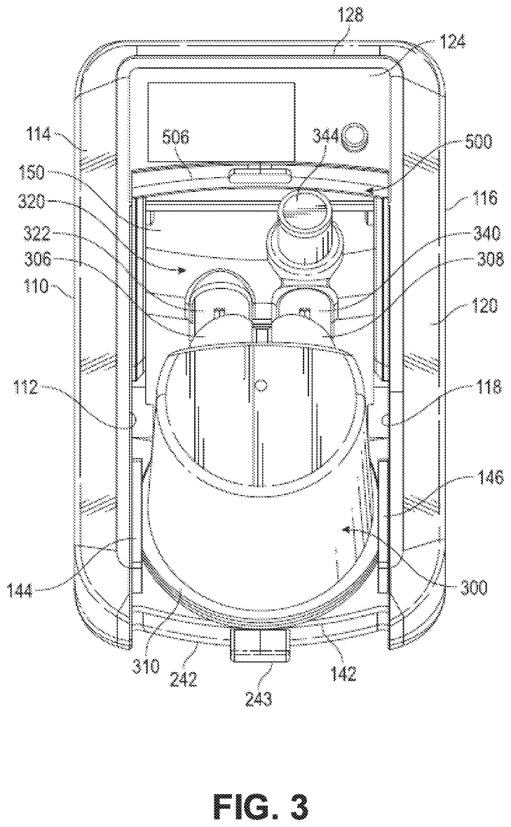

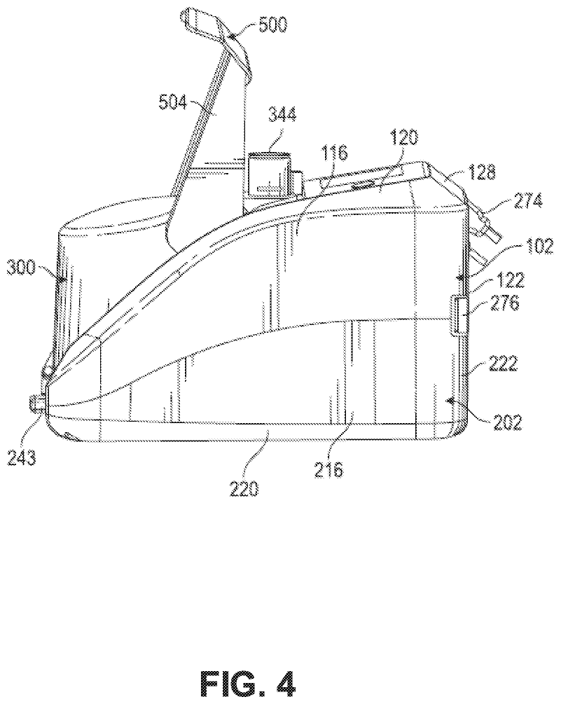

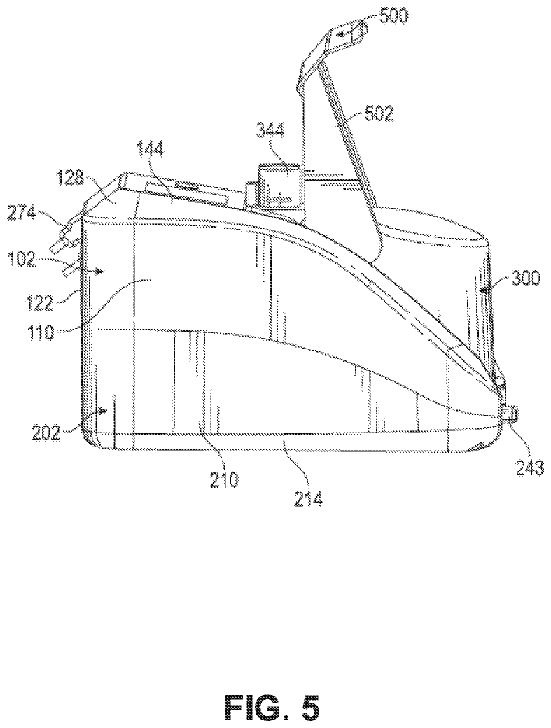

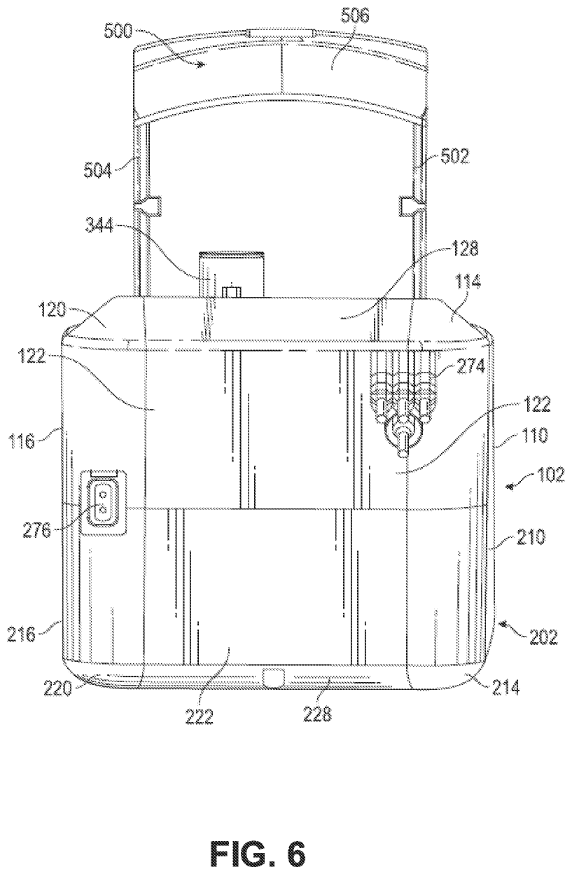

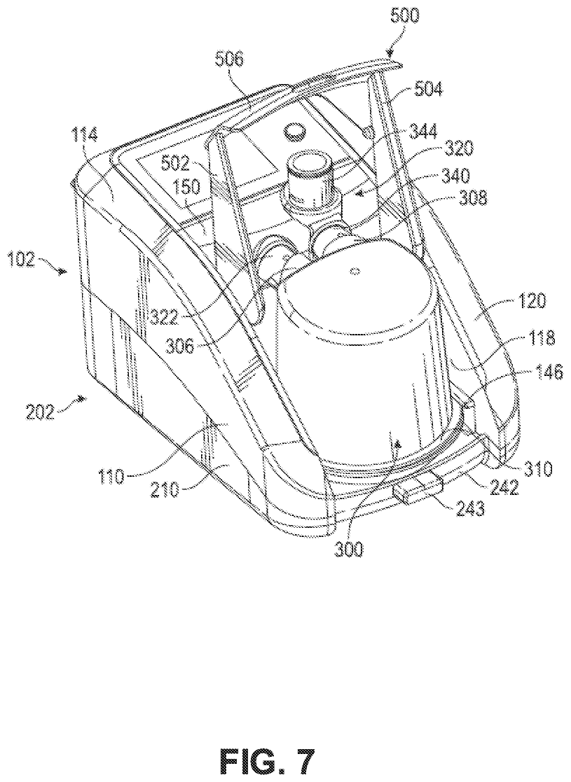

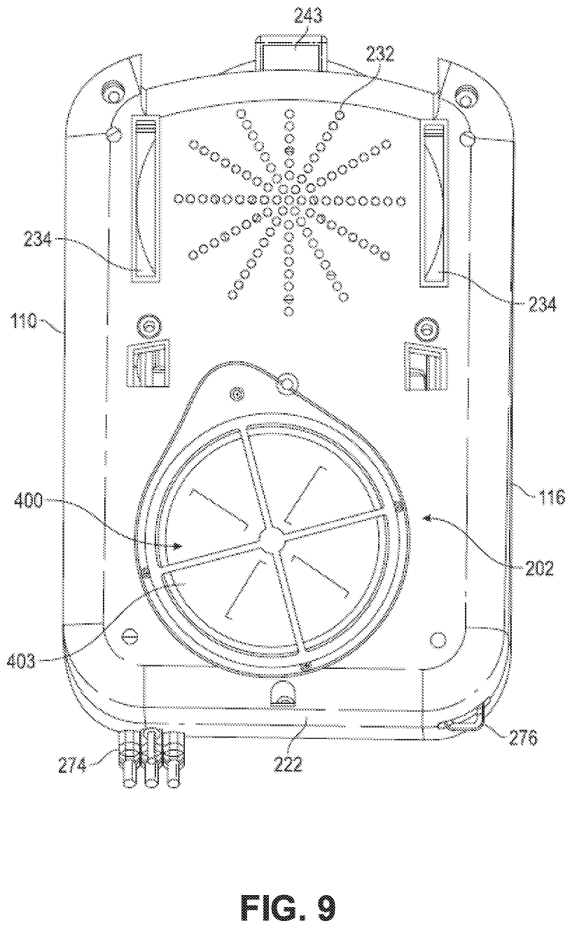

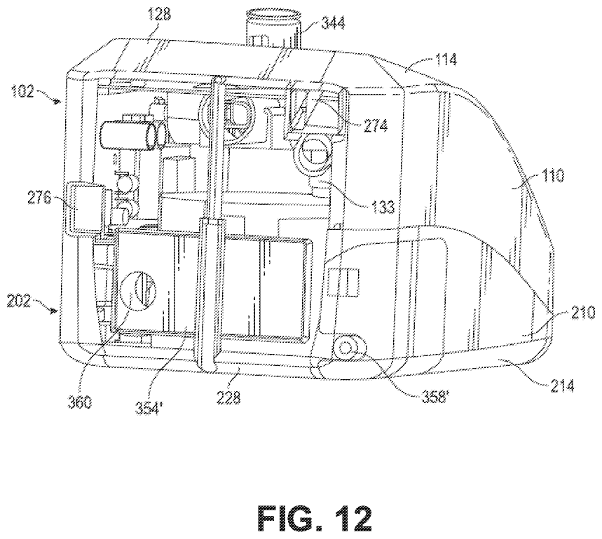

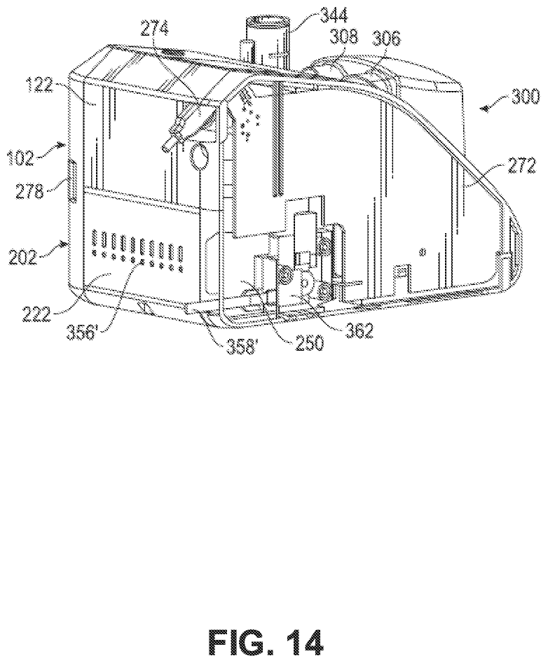

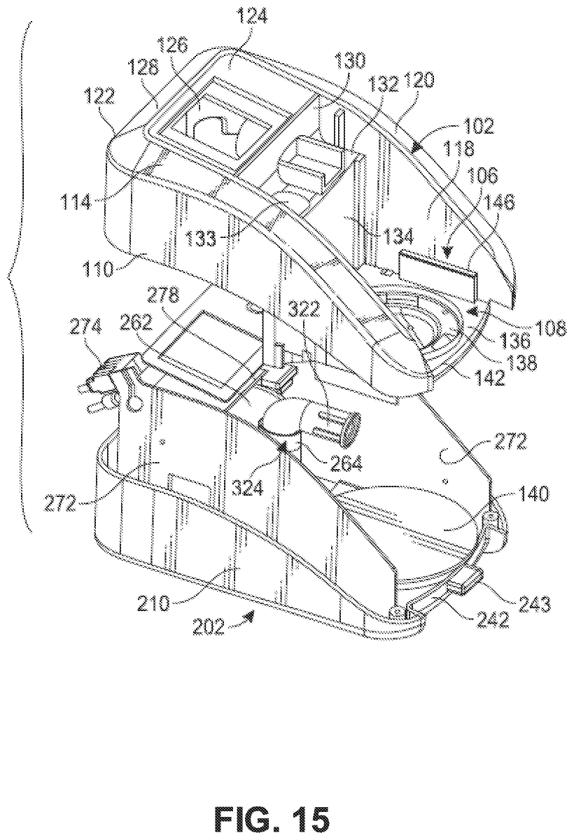

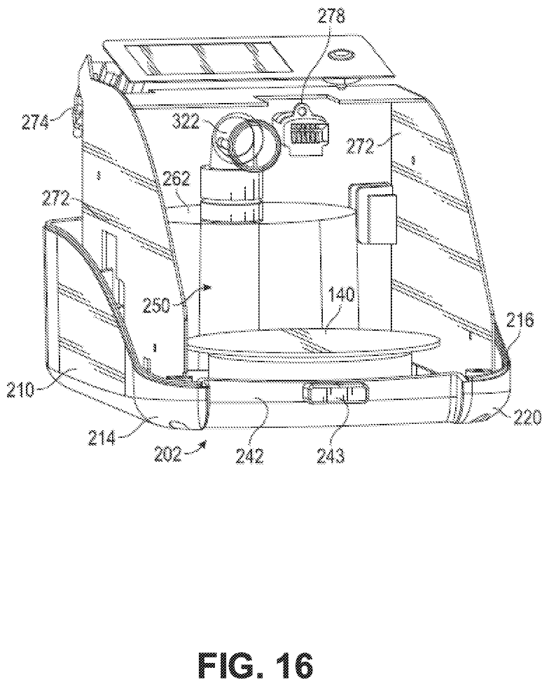

FIGS. 2 to 17B show an example respiratory device of the respiratory system 10 having a main housing 100. The main housing 100 has a main housing upper chassis 102 and a main housing lower chassis 202. The main housing upper chassis 102 has a peripheral wall arrangement 106 (see FIG. 15). The peripheral wall arrangement defines a humidifier or humidification chamber bay 108 for receipt of a removable humidification chamber 300. The removable humidification chamber 300 contains a suitable liquid such as water for humidifying gases that can be delivered to a patient.

In the form shown, the peripheral wall arrangement 106 of the main housing upper chassis 102 can include a substantially vertical left side outer wall 110 that is oriented in a front-to-rear direction of the main housing 100, a substantially vertical left side inner wall 112 that is oriented in a front-to-rear direction of the main housing 100, and an interconnecting wall 114 that extends between and interconnects the upper ends of the left side inner and outer walls 110, 112. The main housing upper chassis 102 can further include a substantially vertical right side outer wall 116 that is oriented in a front-to-rear direction of the main housing 100, a substantially vertical right side inner wall 118 that is oriented in a front-to-rear direction of the main housing 100, and an interconnecting wall 120 that extends between and interconnects the upper ends of the right side inner and outer walls 116, 118. The interconnecting walls 114, 120 are angled towards respective outer edges of the main housing 100, but can alternatively be substantially horizontal or inwardly angled.

The main housing upper chassis 102 can further include a substantially vertical rear outer wall 122. An upper part of the main housing upper chassis 102 can include a forwardly angled surface 124. The surface 124 can have a recess 126 for receipt of a display and user interface module 14. The display can be configured to display characteristics of sensed gas(es) in real time. The system can display the patient detection status of the patient interface. If the patient is not detected, the controller may not output or can stop outputting the respiratory rate value(s) and/or other parameters for display. The controller can also optionally output a message for display that no patient is detected at block 2708. An example of the message can be an icon. An interconnecting wall 128 can extend between and interconnect the upper end of the rear outer wall 122 and the rear edge of the surface 124.

A substantially vertical wall portion 130 can extend downwardly from a front end of the surface 124. A substantially horizontal wall portion 132 can extend forwardly from a lower end of the wall portion 130 to form a ledge. A substantially vertical wall portion 134 can extend downwardly from a front end of the wall portion 132 and terminate at a substantially horizontal floor portion 136 of the humidification chamber bay 108. The left side inner wall 112, right side inner wall 118, wall portion 134, and floor portion 136 together can define the humidification chamber bay 108. The floor portion 136 of the humidification chamber bay 108 can have a recess 138 to receive a heater arrangement such as a heater plate 140 or other suitable heating element(s) for heating liquid in the humidification chamber 300 for use during a humidification process.

The main housing lower chassis 202 can be attachable to the upper chassis 102, either by suitable fasteners or integrated attachment features such as clips for example. The main housing lower chassis 202 can include a substantially vertical left side outer wall 210 that is oriented in a front-to-rear direction of the main housing 100 and is contiguous with the left side outer wall 110 of the upper chassis 102, and a substantially vertical right side outer wall 216 that is oriented in a front-to-rear direction of the main housing 100 and is contiguous with the right side outer wall 116 of the upper chassis 102. The main housing lower chassis 202 can further include a substantially vertical rear outer wall 222 that is contiguous with the rear outer wall 122 of the upper chassis 102.

The lower housing chassis 202 can have a lip 242 that is contiguous with the lip 142 of the upper housing chassis 102, and also forms part of the recess for receiving the handle portion 506 of the lever 500. The lower lip 242 can include a forwardly directed protrusion 243 that acts as a retainer for the handle portion 506 of the lever 500. Instead of the lever 500, the system can have a spring-loaded guard to retain the humidification chamber 300 in the humidification chamber bay 108.

An underside of the lower housing chassis 202 can include a bottom wall 230. Respective interconnecting walls 214, 220, 228 can extend between and interconnect the substantially vertical walls 210, 216, 222 and the bottom wall 230. The bottom wall 230 can include a grill 232 comprising a plurality of apertures to enable drainage of liquid in case of leakage from the humidification chamber 300 (e.g. from spills). The bottom wall 230 additionally can include elongated forward-rearward oriented slots 234. The slots 234 can additionally enable drainage of liquid in case of leakage from the humidification chamber 300, without the liquid entering the electronics housing. In the illustrated configuration, the slots 234 can be wide and elongate relative to the apertures of the grill 232 to maximize the drainage of liquid.

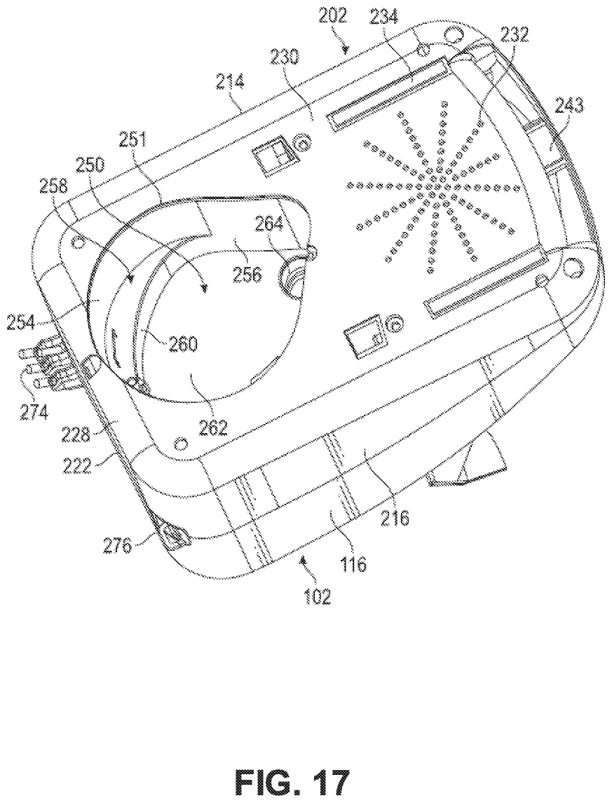

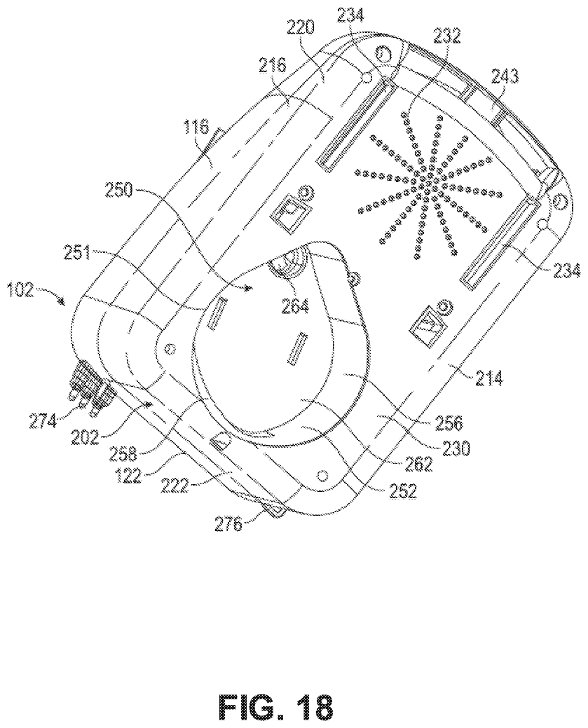

As shown in FIGS. 17 to 18, the lower chassis 202 can have a motor recess 250 for receipt of a motor and sensor module. The motor and sensor module may be non-removable from the main housing 100. The motor and sensor module can be removable from the main housing 100, as illustrated in FIGS. 17-18. A recess opening 251 can be provided in the bottom wall 230 adjacent a rear edge thereof, for receipt of a motor/sensor module. A continuous, gas impermeable, unbroken peripheral wall 252 can be integrally formed with the bottom wall 230 of the lower chassis 202 and extend upwardly from the periphery of the opening 251. A rearward portion 254 of the peripheral wall 252 has a first height, and a forward portion 256 of the peripheral wall 252 has a second height that is greater than the first height. The rearward portion 254 of the peripheral wall 252 terminates at a substantially horizontal step 258, which in turn terminates at an upper auxiliary rearward portion 260 of the peripheral wall 252. The forward portion 256 and upper auxiliary rearward portion 260 of the peripheral wall 252 terminate at a ceiling 262. All of the walls and the ceiling 262 can be continuous, gas impermeable, and unbroken other than the gases flow passage. Therefore, the entire motor recess 250 can be gas impermeable and unbroken, other than the gases flow passage.

The motor and sensor module can be insertable into the recess 250 and attachable to the lower chassis 202. Upon insertion of the motor and sensor module into the lower chassis 202, the gases flow passage tube 264 can extend through the downward extension tube 133 and be sealed by the soft seal.

The humidification chamber 300 can be fluidly coupled to the apparatus 10 in a linear slide-on motion in a rearward direction of the humidification chamber 300 into the chamber bay 108, from a position at the front of the housing 100 in a direction toward the rear of the housing 100. A gases outlet port 322 can be in fluid communication with the motor. Humidity is advantageous as it provides airway hydration, improves comfort and maintains physiological stability in compromised airways.

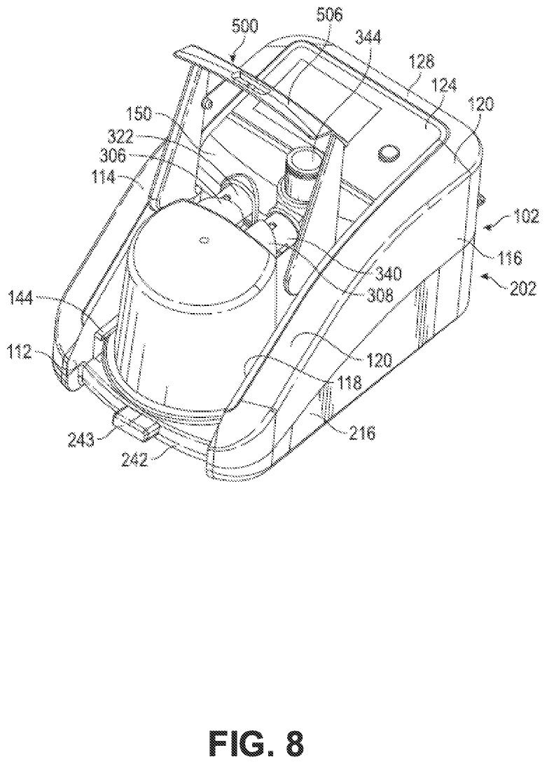

A gases inlet port 340 (humidified gases return) as shown in FIG. 8 can include a removable L-shaped elbow. The removable elbow can further include a patient outlet port 344 for coupling to the patient conduit 16 to deliver gases to the patient interface. The gases outlet port 322, gases inlet port 340, and patient outlet port 344 each can have soft seals such as O-ring seals or T-seals to provide a sealed gases passageway between the apparatus 10, the humidification chamber 300, and the patient conduit 16.

The humidification chamber gases inlet port 306 can be complementary with the gases outlet port 322, and the humidification chamber gases outlet port 308 can be complementary with the gases inlet port 340. The axes of those ports can be parallel to each other to enable the humidification chamber 300 to be inserted into the chamber bay 108 in a linear movement.

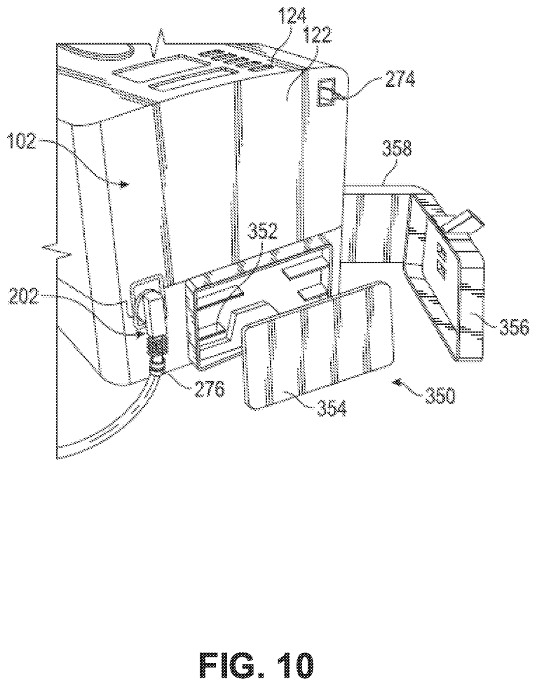

The respiratory device can have air and oxygen (or alternative auxiliary gas) inlets in fluid communication with the motor to enable the motor to deliver air, oxygen (or alternative auxiliary gas), or a mixture thereof to the humidification chamber 300 and thereby to the patient. As shown in FIG. 10, the device can have a combined air/oxygen (or alternative auxiliary gas) inlet arrangement 350. This arrangement can include a combined air/oxygen port 352 into the housing 100, a filter 354, and a cover 356 with a hinge 358. A gases tube can also optionally extend laterally or in another appropriate direction and be in fluid communication with an oxygen (or alternative auxiliary gas) source. The port 352 can be fluidly coupled with the motor 402. For example, the port 352 may be coupled with the motor/sensor module 400 via a gases flow passage between the port 352 and an inlet aperture or port in the motor and sensor module 400, which in turn would lead to the motor.

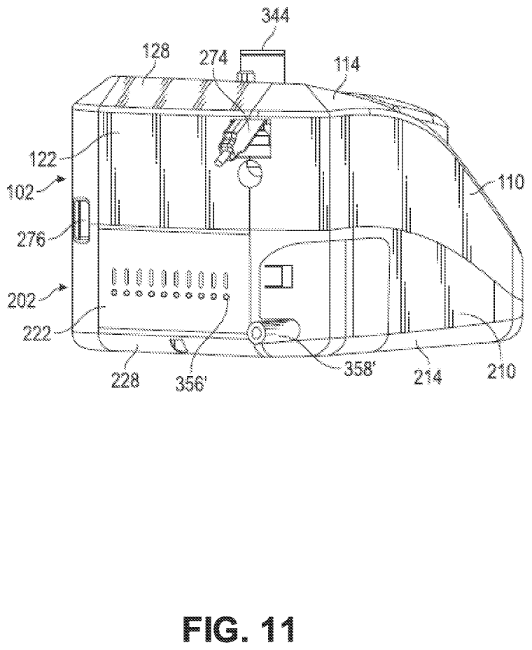

The device can have the arrangement shown in FIGS. 11 to 14 to enable the motor to deliver air, oxygen (or alternative auxiliary gas), or a suitable mixture thereof to the humidification chamber 300 and thereby to the patient. This arrangement can include an air inlet 356′ in the rear wall 222 of the lower chassis 202 of the housing 100. The air inlet 356′ comprises a rigid plate with a suitable grill arrangement of apertures and/or slots. Sound dampening foam may be provided adjacent the plate on the interior side of the plate. An air filter box 354′ can be positioned adjacent the air inlet 356′ internally in the main housing 100, and include an air outlet port 360 to deliver filtered air to the motor via an air inlet port 404 in the motor/sensor module 400. The air filter box 354′ may include a filter configured to remove particulates (e.g. dust) and/or pathogens (e.g. viruses or bacteria) from the gases flow. A soft seal such as an O-ring seal can be provided between the air outlet port 360 and air inlet port 404 to seal between the components. The device can include a separate oxygen inlet port 358′ positioned adjacent one side of the housing 100 at a rear end thereof, the oxygen port 358′ for receipt of oxygen from an oxygen source such as a tank or source of piped oxygen. The oxygen inlet port 358′ is in fluid communication with a valve 362. The valve 362 can suitably be a solenoid valve that enables the control of the amount of oxygen that is added to the gases flow that is delivered to the humidification chamber 300. The oxygen port 358′ and valve 362 may be used with other auxiliary gases to control the addition of other auxiliary gases to the gases flow. The other auxiliary gases can include any one or more of a number of gases useful for gas therapy, including but not limited to heliox and nitric oxide.

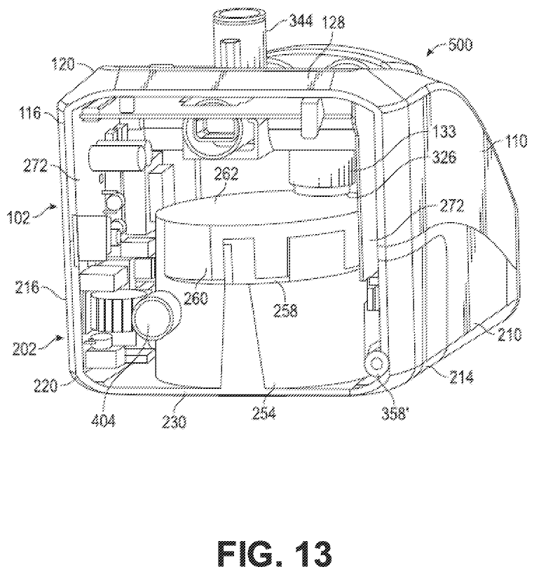

As shown in FIGS. 13 to 16, the lower housing chassis 202 can include suitable electronics boards, such as sensing circuit boards. The electronics boards can be positioned adjacent respective outer side walls 210, 216 of the lower housing chassis 202. The electronics boards can contain, or can be in electrical communication with, suitable electrical or electronics components, such as but not limited to microprocessors, capacitors, resistors, diodes, operational amplifiers, comparators, and switches. Sensors can be used with the electronic boards. Components of the electronics boards (such as but not limited to one or more microprocessors) can act as the controller 13 of the apparatus.

One or both of the electronics boards can be in electrical communication with the electrical components of the apparatus 10, including the display unit and user interface 14, motor, valve 362, and the heater plate 140 to operate the motor to provide the desired flow rate of gases, operate the humidification chamber 300 to humidify and heat the gases flow to an appropriate level, and supply appropriate quantities of oxygen (or quantities of an alternative auxiliary gas) to the gases flow.

The electronics boards can be in electrical communication with a connector arrangement 274 projecting from the rear wall 122 of the upper housing chassis 102. The connector arrangement 274 may be coupled to an alarm, pulse oximetry port, and/or other suitable accessories. The electronics boards can also be in electrical communication with an electrical connector 276 that can also be provided in the rear wall 122 of the upper housing chassis 102 to provide mains or battery power to the components of the device.

As mentioned above, operation sensors, such as flow, temperature, humidity, and/or pressure sensors can be placed in various locations in the respiratory device, the patient breathing conduit 16, and/or cannula 17 such as shown in FIG. 1A. The electronics boards can be in electrical communication with those sensors. Output from the sensors can be received by the controller 13, to assist the controller 13 to operate the respiratory system 10 in a manner that provides optimal therapy, including meeting inspiratory demand.

As outlined above, the electronics boards and other electrical and electronic components can be pneumatically isolated from the gases flow path to improve safety. The sealing also prevents water ingress.

### Control System

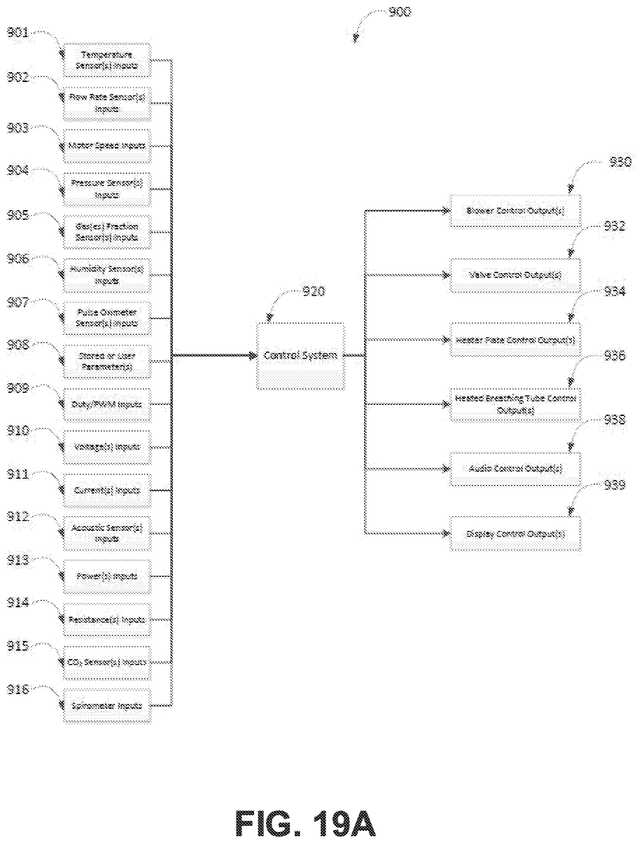

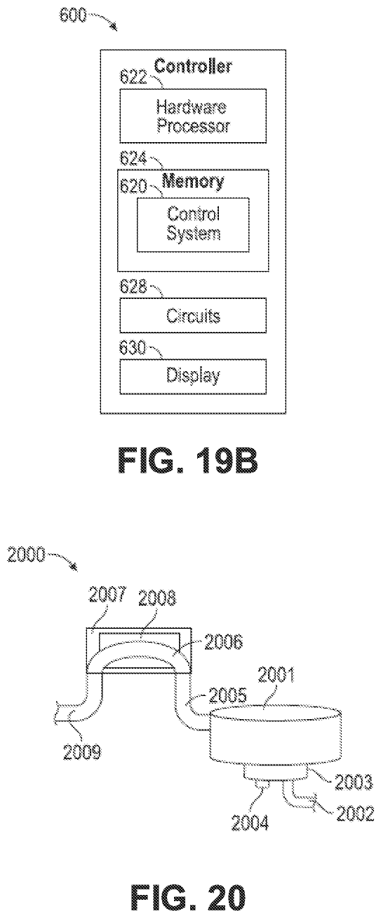

FIG. 19A illustrates a block diagram 900 of an example control system 920 (which can be the controller 13 in FIG. 1A) that can detect patient conditions and control operation of the respiratory system including the gases source. The control system 920 can manage a flow rate of the gases flowing through the respiratory system as is the gases are delivered to a patient. For example, the control system 920 can increase or decrease the flow rate by controlling an output of a motor speed of the blower (hereinafter also referred to as a “blower motor”) 930 or an output of a valve 932 in a blender. The control system 920 can automatically determine a set value or a personalized value of the flow rate for a particular patient as discussed below. The flow rate can be optimized by the control system 920 to improve patient comfort and therapy.

The control system 920 can also generate audio and/or display/visual outputs 938, 939. For example, the flow therapy apparatus can include a display and/or a speaker. The display can indicate to the physicians any warnings or alarms generated by the control system 920. The display can also indicate control parameters that can be adjusted by the physicians. For example, the control system 920 can automatically recommend a flow rate for a particular patient. The control system 920 can also determine a respiratory state of the patient, including but not limited to generating a respiratory rate of the patient, and send it to the display, which will be described in greater detail below.

The control system 920 can change heater control outputs to control one or more of the heating elements (for example, to maintain a temperature set point of the gases delivered to the patient). The control system 920 can also change the operation or duty cycle of the heating elements. The heater control outputs can include heater plate control output(s) 934 and heated breathing tube control output(s) 936.

The control system 920 can determine the outputs 930-939 based on one or more received inputs 901-916. The inputs 901-916 can correspond to sensor measurements received automatically by the controller 600 (shown in FIG. 19B). The control system 920 can receive sensor inputs including but not limited to temperature sensor(s) inputs 901, flow rate sensor(s) inputs 902, motor speed inputs 903, pressure sensor(s) inputs 904, gas(s) fraction sensor(s) inputs 905, humidity sensor(s) inputs 906, pulse oximeter (for example, SpC) sensor(s) inputs 907, stored or user parameter(s) 908, duty cycle or pulse width modulation (PWM) inputs 909, voltage(s) inputs 910, current(s) inputs 911, acoustic sensor(s) inputs 912, power(s) inputs 913, resistance(s) inputs 914, optional CO₂ sensor(s) inputs 915, and/or optional spirometer inputs 916. The pulse oximeter may also be optional. The control system 920 can receive inputs from the user or stored parameter values in a memory 624 (shown in FIG. 19B). The control system 920 can dynamically adjust flow rate for a patient over the time of their therapy. The control system 920 can continuously detect system parameters and patient parameters. A person of ordinary skill in the art will appreciate based on the disclosure herein that any other suitable inputs and/or outputs can be used with the control system 920.

### Controller

FIG. 19B illustrates a block diagram of a controller 600 (which can be the controller 13 in FIG. 1A or the controller in FIG. 1B). The controller 600 can include programming instructions for detection of input conditions and control of output conditions. The programming instructions can be stored in the memory 624 of the controller 600. The programming instructions can correspond to the methods, processes and functions described herein. The programming instructions can be executed by one or more hardware processors 622 of the controller 600. The programming instructions can be implemented in C, C++, JAVA, or any other suitable programming languages. Some or all of the portions of the programming instructions can be implemented in application specific circuitry 628 such as ASICs and FPGAs.

The controller 600 can also include circuits 628 for receiving sensor signals. The controller 600 can further include a display 630 for transmitting status of the patient and the respiratory assistance system. The display 630 can also show warnings and/or other alerts. The display 630 can be configured to display characteristics of sensed gas(es) in real time or otherwise. The controller 600 can also receive user inputs via the user interface such as display 630. The user interface can include button(s) and/or dial(s). The user interface can comprise a touch screen.

### Motor and Sensor Module

Any of the features of the respiratory system described herein, including but not limited to the humidification chamber, the flow generator, the user interface, the controller, and the patient breathing conduit configured to couple the gases flow outlet of the respiratory system to the patient interface, can be combined with any of the sensor modules described herein.

FIG. 20 illustrates a block diagram of the motor and sensor module 2000, which can be received by the recess 250 in the respiratory device (shown in FIGS. 17 and 18). The motor and sensor module can include a blower 2001, which entrains room air to deliver to a patient. The blower 2001 can be a centrifugal blower.

One or more sensors (for example, Hall-effect sensors) may be used to measure a motor speed of the blower motor. The blower motor may comprise a brushless DC motor, from which motor speed can be measured without the use of separate sensors. For example, during operation of a brushless DC motor, back-EMF can be measured from the non-energized windings of the motor, from which a motor position can be determined, which can in turn be used to calculate a motor speed. In addition, a motor driver may be used to measure motor current, which can be used with the measured motor speed to calculate a motor torque. The blower motor may comprise a low inertia motor. Room air can enter a room air inlet 2002, which enters the blower 2001 through an inlet port 2003. The inlet port 2003 can include a valve 2004 through which a pressurized gas may enter the blower 2001. The valve 2004 can control a flow of oxygen into the blower 2001. The valve 2004 can be any type of valve, including a proportional valve or a binary valve. In some configurations, the inlet port does not include a valve.

The blower 2001 can operate at a motor speed of greater than 1,000 RPM and less than 30,000 RPM, greater than 2,000 RPM and less than 21,000 RPM, or between any of the foregoing values. Operation of the blower 2001 mixes the gases entering the blower 2001 through the inlet port 2003. Using the blower 2001 as the mixer can decrease the pressure drop that would otherwise occur in a system with a separate mixer, such as a static mixer comprising baffles, because mixing requires energy.

The mixed air can exit the blower 2001 through a conduit 2005 and enters the flow path 2006 in the sensor chamber 2007. A sensing circuit board with sensors 2008 can positioned in the sensor chamber 2007 such that the sensing circuit board is at least partially immersed in the gases flow. At least some of the sensors 2008 on the sensing circuit board can be positioned within the gases flow to measure gases properties within the flow. After passing through the flow path 2006 in the sensor chamber 2007, the gases can exit 2009 to the humidification chamber.

Positioning sensors 2008 downstream of the combined blower and mixer 2001 can increase accuracy of measurements, such as the measurement of gases fraction concentration, including oxygen concentration, over systems that position the sensors upstream of the blower and/or the mixer. Such a positioning can give a repeatable flow profile. Further, positioning the sensors downstream of the combined blower and mixer avoids the pressure drop that would otherwise occur, as where sensing occurs prior to the blower, a separate mixer, such as a static mixer with baffles, is required between the inlet and the sensing system. The mixer can introduce a pressure drop across the mixer. Positioning the sensing after the blower can allow the blower to be a mixer, and while a static mixer would lower pressure, in contrast, a blower increases pressure. Also, immersing at least part of the sensing circuit board and sensors 2008 in the flow path can increase the accuracy of measurements because the sensors being immersed in the flow means they are more likely to be subject to the same conditions, such as temperature and pressure, as the gases flow and therefore provide a better representation of the gases flow characteristics.

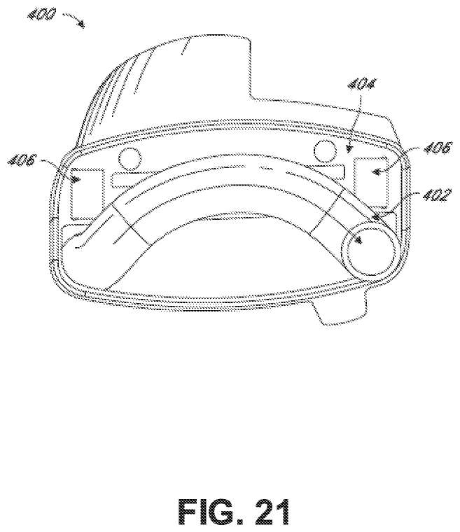

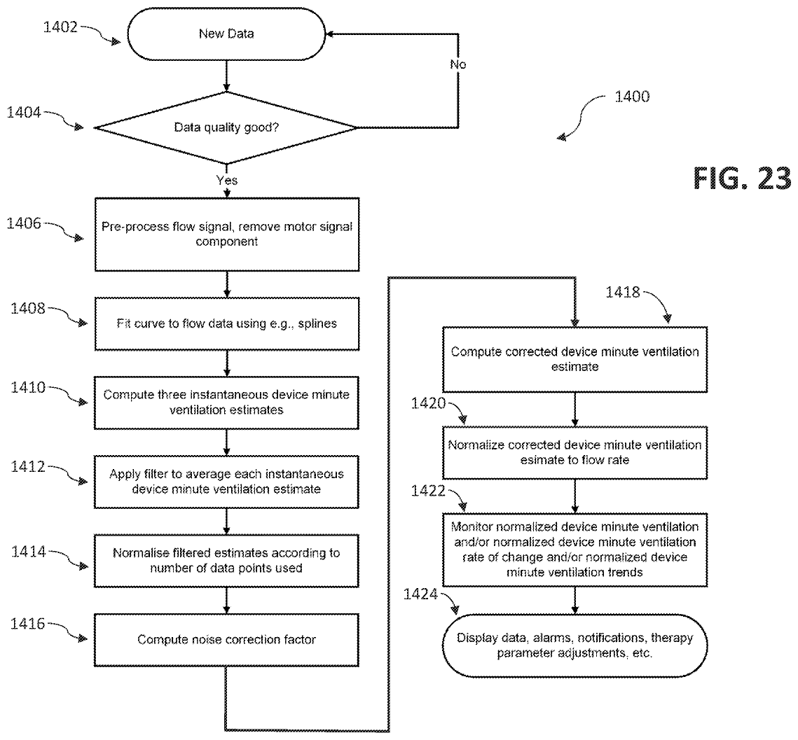

Turning to FIG. 21, the gases exiting the blower can enter a flow path 402 in a sensor chamber 400, which can be positioned within the motor and sensor module and can be the sensor chamber 2007 of FIG. 20. The flow path 402 can have a curved shape. The flow path 402 can be configured to have a curved shape with no sharp turns. The flow path 402 can have curved ends with a straighter section between the curved ends. A curved flow path shape can reduce pressure drop in a gases flow without reducing the sensitivity of flow measurements by partially coinciding a measuring region with the flow path to form a measurement portion of the flow path, which will be described below with reference to FIGS. 23A-23B.

A sensing circuit board 404 with sensors, such as acoustic transmitters and/or receivers, humidity sensor, temperature sensor, thermistor, and the like, can be positioned in the sensor chamber 400 such that the sensing circuit board 404 is at least partially immersed in the flow path 402. Immersing at least part of the sensing circuit board and sensors in the flow path can increase the accuracy of measurements because the sensors immersed in the flow are more likely to be subject to the same conditions, such as temperature and pressure, as the gases flow, and therefore provide a better representation of the characteristics of the gases flow. After passing through the flow path 402 in the sensor chamber 400, the gases can exit to the humidification chamber.

The gases flow rate may be measured using at least two different types of sensors. The first type of sensor can comprise a thermistor, which can determine a flow rate by monitoring heat transfer between the gases flow and the thermistor. The thermistor flow sensor can run the thermistor at a constant target temperature within the flow when the gases flow around and past the thermistor. The sensor can measure an amount of power required to maintain the thermistor at the target temperature. The target temperature can be configured to be higher than a temperature of the gases flow, such that more power is required to maintain the thermistor at the target temperature at a higher flow rate.

The thermistor flow rate sensor can also maintain a plurality of (for example, two, three, or more) constant temperatures on a thermistor to avoid the difference between the target temperature and the gases flow temperature from being too small or too large. The plurality of different target temperatures can allow the thermistor flow rate sensor to be accurate across a large temperature range of the gases. For example, the thermistor circuit can be configured to be able to switch between two different target temperatures, such that the temperature of the gases flow will always fall within a certain range relative to one of the two target temperatures (for example, not too close but not too far). The thermistor circuit can be configured to operate at a first target temperature of about 50° ° C. to about 70° ° C., or about 66° C. The first target temperature can be associated with a desirable flow temperature range of between about 0° ° C. to about 60° C., or about 0° C. and about 40° C. The thermistor circuit can be configured to operate at a second target temperature of about 90° C. to about 110° C., or about 100° C. The second target temperature can be associated with a desirable flow temperature range of between about 20° ° C. to about 100° ° C., or about 30° C. and about 70° C.

The controller can be configured to adjust the thermistor circuit to change between at least the first and second target temperature modes by connecting or bypassing a resistor within the thermistor circuit. The thermistor circuit can be arranged as a Wheatstone bridge configuration comprising a first voltage divider arm and a second voltage divider arm. The thermistor can be located on one of the voltage divider arms. More details of a thermistor flow rate sensor are described in PCT Application No. PCT/NZ2017/050119, filed Sep. 3, 2017, which is incorporated by reference herein in its entirety.

The second type of sensor can comprise an acoustic sensor assembly. Acoustic sensors including acoustic transmitters and/or receivers can be used to measure a time of flight of acoustic signals to determine gases velocity and/or composition, which can be used in flow therapy apparatuses. In one ultrasonic sensing (including ultrasonic transmitters and/or receivers) topology, a driver causes a first sensor, such as an ultrasonic transducer, to produce an ultrasonic pulse in a first direction. A second sensor, such as a second ultrasonic transducer, receives this pulse and provides a measurement of the time of flight of the pulse between the first and second ultrasonic transducers. Using this time of flight measurement, the speed of sound of the gases flow between the ultrasonic transducers can be calculated by a processor or controller of the respiratory system. The second sensor can transmit and the first sensor can receive a pulse in a second direction opposite the first direction to provide a second measurement of the time of flight, allowing characteristics of the gases flow, such as a flow rate or velocity, to be determined. In another acoustic sensing topology, acoustic pulses transmitted by an acoustic transmitter, such as an ultrasonic transducer, can be received by acoustic receivers, such as microphones. More details of an acoustic flow rate sensor are described in PCT Application PCT/NZ2016/050193, filed Dec. 2, 2016, which is incorporated by reference herein in its entirety.

Readings from both the first and second types of sensors can be combined to determine a more accurate flow measurement. For example, a previously determined flow rate and one or more outputs from one of the types of sensor can be used to determine a predicted current flow rate. The predicted current flow rate can then be updated using one or more outputs from the other one of the first and second types of sensor, in order to calculate a final flow rate.

### Pre-Processing Data

As discussed above, the flow data in an unsealed system, such as nasal high flow systems, can be difficult to determine. The open nature of the system results in a very low signal to noise ratio. Any flow data measured can include various irregularities and noise that can obscure the flow data and which must be accounted for to accurately determine the desired measurement. Flow data is important as it can be informative of the unsealed system and patient breath flow.

In order to remove noise and other irregularities from any obtained flow data, the flow signal can be fed through a pre-processing step. Pre-processing can allow the controller to remove certain distortions from the flow parameters, such that the flow parameter signal that is used to determine the device output and/or patient breathing parameters (e.g., nasal minute ventilation and/or peak inspiratory flow) can better reflect the effect the gas flow parameter used in the patient's treatment is having on the patient's respiration. More details of pre-processing of flow signals are described in PCT Application PCT/IB2020/051816, filed Mar. 4, 2020, which is incorporated by reference herein in its entirety.

If the patient is attached to the respiratory system and breathing through the patient interface, fluctuations in pre-processed flow rate or other flow parameter data obtained in an open system are made up of random uncorrelated noise and a correlated breathing signal generated from various sources. The pre-processing of the data can start with the controller receiving the flow parameter data (such as unprocessed data). The controller can then perform the pre-processing step, for example, by determining if the flow parameter data is good or suitable for use. If the data is not suitable for use, the controller can discard the data.

In determining the suitability of the data, the controller can receive second flow parameter data that is of a different type than a first flow parameter data. The second flow parameter data is assumed to have some correlation to the first parameter. The second flow parameter data can include, for example, the motor speed, pressure, and/or oxygen flow rate or concentration or any other parameter that can have an effect on or provide an indication of the gases flow rate that is separate from the effect of the patient's respiration on the gases flow rate. The controller can be configured to determine whether the second flow parameter data is useful as a correlation parameter to the first flow parameter data. For example, the second flow parameter data can be a useful correlation metric if the second flow parameter data meets a threshold level. If the second flow parameter data does not meet a threshold level, then it is assumed the second flow parameter data is not correlated to the first flow parameter data. As such, the second flow parameter data can be ignored or thrown out (i.e., deleted or not utilized by the controller). If there is insufficient second flow parameter data, the controller can determine that it has insufficient data to use the first flow parameter data and may discard the first parameter data. If the second flow parameter data meets a minimum threshold level, the controller can determine that the first parameter data is suitable for use.

As an example, the second flow parameter data can represent the motor speed. In order to identify the patient's respiration in the first flow parameter data, the motor needs to be operating at a sufficient speed. If the motor speed is too low, the effect or correlation of the motor speed on the flow data (such as the flow rate) may not be accurately predictable. Therefore, after the controller has received the motor speed data, the controller can compare the motor speed to a minimum motor speed threshold. If the motor speed is below the threshold, the controller can deem the first flow parameter data as unsuitable and can discard a portion or all of the first flow parameter data. However, if the motor speed is above the threshold, the controller can calculate the recent changes in the motor speed. A change in motor speed can result in a change in the first flow parameter data, which makes it more difficult to identify the patient's respiration in the first flow parameter data. While the effect of the motor speed can be removed from the first flow parameter data to some degree, larger changes in motor speed may make the data too unreliable for identifying the patient's respiration. Therefore, the controller can apply a running filter to the relative changes in motor speed in order to generate a first value representing the recent relative changes in motor speed. The controller can then compare the first value with a first threshold. If the first value is above the first threshold, the controller can deem the flow parameter data to be unsuitable, and the flow data point can be discarded. If the first value is below the first threshold, the controller can deem the flow parameter data to be suitable for use.

As another example, the second flow parameter data can represent the concentration of a supplementary gas from a supplementary gas source. The first flow parameter data (such as the flow rate) can be affected by the flow rate or concentration of supplementary gas from a supplementary gas source. The controller can receive an oxygen flow rate data or an oxygen concentration data. The controller can calculate recent changes in the oxygen flow rate or the oxygen concentration. If the flow rate or concentration of oxygen changes, the resulting change in the total flow rate can make it more difficult to identify the patient's respiration in the flow rate signal or other flow parameter signal. The controller can therefore apply a running filter to the changes in oxygen concentration of the gases or the oxygen flow rate in order to generate a second value representing the recent changes in oxygen concentration or flow rate. The controller can compare the second value with a second threshold. If the second value is above the second threshold, the controller can determine the first flow parameter data is unsuitable, and the first flow parameter data point can be discarded. However, if the second flow parameter data is below the threshold, the controller can deem the flow parameter data to be suitable.

As described above, if the controller deems the data to be suitable, the first flow parameter data (or any other flow parameter data) can also be modified to remove the effect of the motor (or other factors, such as the oxygen concentration or flow rate). Modifying the first flow parameter data can involve removing the assumed effect of other variables from the first flow parameter data (such as the motor speed). This assumed effect is only valid if the gases flow parameter data meets certain criteria. As described above, if these criteria are not met, the data may be discarded.

The process can modify the first flow parameter data to remove the effect of motor speed. The effect of the motor can be estimated using the motor speed and the flow conductance. The controller can measure an instantaneous flow conductance. The flow conductance can be calculated as provided below:

$$C = filt ( \frac{Q}{{ω}_{motor}} )$$

In the equation provided above, C is the flow conductance, filt( ) is a filter function, Q is the flow parameter data, and ωₘₒₜₒᵣ is the motor speed. In some configurations, the filter function is a low-pass filter. In some examples, the flow parameter data is a flow rate signal generated by the device flow sensor. The flow conductance is approximately constant with time, and can therefore be estimated using a low pass filter. The controller measures the instantaneous flow conductance at each iteration using the current motor speed and a measured flow rate. The controller can filter the instantaneous flow conductance in order to determine a filtered flow conductance.

The controller can compare the instantaneous flow conductance with the filtered flow conductance to see if the difference is significantly different. If the difference is significant, it is likely that something has changed the physical system, such as the cannula being attached or detached. The instantaneous flow conductance can be compared with the filtered flow conductance by taking the difference of the two variables and comparing it with a minimum or a maximum threshold. If the difference exceeds or falls below the threshold, the difference is considered to be significant, and the controller can reset the filtered flow conductance. The controller can also vary the filter coefficient of the filter function in the filtered flow conductance calculation based on the difference between the instantaneous flow conductance and the filtered flow conductance. This allows the filtered flow conductance to change more quickly when the variance of the flow conductance is high, such as when the cannula has first been attached.

If the difference between the instantaneous flow conductance with the filtered flow conductance does not exceed the threshold, the difference is considered to be not significant, and the controller can estimate the effect of the motor on the flow rate. The controller can output a value of the effect using the filtered flow conductance and the motor speed. The value can be subtracted or otherwise removed from the flow rate data to arrive at the pre-processed flow rate data. The pre-processed flow rate data can be more indicative of the patient's respiratory flow (although the pre-processed flow rate data can still include signal noise).

The controller can also track the recent changes in the flow conductance. The changes can be tracked by adding the difference between the last two instantaneous flow conductance values to a running total, which is then decayed over time. The decayed running total is filtered to obtain the filtered recent changes in flow conductivity. The filtered recent changes in flow conductivity can be used in further parts of the frequency analysis algorithm along with the pre-processed flow rate data.

### Nasal Minute Ventilation & Peak Inspiratory Flow

As discussed above, while inhalation and exhalation are relatively easy to measure in sealed systems, breathing parameters are much more difficult to measure in unsealed systems. In unsealed systems, such as a nasal high flow system, the open nature of the system (due to use of an unsealed patient interface) makes it significantly more difficult to determine patient inhalation and exhalation because the desired signal is often weak and obscured by noise.

The present disclosure provides reliable methods of estimating key patient breathing parameters in an unsealed system (e.g., unsealed nasal cannula used in a high flow system). The controller can be configured to estimate nasal minute ventilation (MVₙ) and estimate peak inspiratory flow (ûₚₑₐₖ). Nasal minute ventilation (MVₙ) is a measurement that is created specifically for the context of the present method for estimating breathing parameters in an unsealed system. As a concept, nasal minute ventilation (MVₙ) may be similar to a conventional minute ventilation, however it is measure exclusively in relation to the nasal cavity (i.e., the volume of gas entering or exiting the nasal cavity per minute). Because of the open nature in a nasal high flow system, the nasal minute ventilation (MVₙ) is distinct from conventional measurements of minute ventilation and nasal minute ventilation (MVₙ) should not be considered equivalent to conventional minute ventilation. The disclosed processes of performing an analysis of gases flow parameter to obtain an estimate of the nasal minute ventilation (MVₙ) and the peak inspiratory flow (ûₚₑₐₖ) can provide reliable methods of estimating key patient breathing parameters when using unsealed nasal systems. Furthermore, the rate of change and/or trends of the nasal minute ventilation (MVₙ) and the peak inspiratory flow (ûₚₑₐₖ) can be monitored as opposed to (or in addition to) the actual values. The monitoring of the change and/or trends of the nasal minute ventilation (MVₙ) and the peak inspiratory flow (ûₚₑₐₖ) can provide a robust indicator of nasal high flow therapy efficacy that is significantly less subject to error.

The ability to estimate the value of the nasal minute ventilation (MVₙ) and/or the peak inspiratory flow (ûₚₑₐₖ) and/or monitor the rate of change of each of the aforementioned values can enable new functions in unsealed high flow systems. Estimated breathing parameters can provide valuable indicator(s) of therapy efficacy and help to improve therapy outcomes. For example, the ability to calculate and monitor the change of the nasal minute ventilation (MVₙ) and the peak inspiratory flow (ûₚₑₐₖ) in an unsealed nasal high flow system can allow a user or health care professional to adjust therapy parameters (e.g., device flow rate) or to change the patient interface when a patient is not responding positively to the current therapy parameters. In some configurations, knowledge of the change of the nasal minute ventilation (MVₙ) and the peak inspiratory flow (ûₚₑₐₖ) in an unsealed nasal high flow system can allow a user or health care professional to more appropriately, precisely, and/or reliably change therapy parameters (e.g., device flow rate) or to change the patient interface when a patient is not responding positively to the current therapy parameters. In some examples, a frequent problem encountered in unsealed nasal high flow systems is knowing whether sufficient (e.g., therapeutically effective) flow rate being set by users or health care professionals. The calculation of the estimated nasal minute ventilation (MVₙ) and/or the peak inspiratory flow (ûₚₑₐₖ) and the associated trend of these values can help to provide a feedback loop that allows a user or health care professional to adjust the flow rate while therapy is being provided. The unsealed nasal high flow system can also be configured to automatically adjust the flow rate in a closed feedback loop. In some configurations, the nasal minute ventilation (MVₙ) and/or the peak inspiratory flow (ûₚₑₐₖ) can be passed as inputs to a closed-loop feedback control system to adjust flow rate or other therapy parameters. In some examples, additional inputs can be passed to the closed-loop feedback control system to adjust flow rates or other therapy parameters. These can include, for example, the values of the nasal minute ventilation (MVₙ) and/or the peak inspiratory flow (ûₚₑₐₖ), the rates of change of the nasal minute ventilation (MVₙ) and/or the peak inspiratory flow (ûₚₑₐₖ), the trends of the nasal minute ventilation (MVₙ) and/or the peak inspiratory flow (ûₚₑₐₖ), and/or other values representative of the nasal minute ventilation (MVₙ) and/or the peak inspiratory flow (Ûpeak).

Information regarding the estimated nasal minute ventilation (MVₙ) and/or the peak inspiratory flow (ûₚₑₐₖ) can be used to provide alarms, notifications, and/or indicators that are configured to trigger based on certain thresholds indicating therapy ineffectiveness. This can provide users and health care professionals with more information regarding patient health and nasal high flow therapy efficacies. This can encourage or enable more accurate adjustment of therapy parameters, leading to improved outcomes.

### Nasal Minute Ventilation

The controller can include processes that are configured to calculate an estimate of nasal minute ventilation (MVₙ). The device minute ventilation (MV_{device}) represents the device minute ventilation, a measure of the average volume of air being pushed in and out of the device per minute. Because of the high-leakage nature of nasal cannula interfaces in unsealed systems, it can be very difficult to accurately determine the true minute ventilation of a patient. This is particularly true while high flow therapy is being provided. An estimation of the nasal minute ventilation (MVₙ) can therefore be calculated by using manual inputs characterizing the fit of the cannula (e.g., cannula size, model dimensions) and a known constant flow rate of gases through the cannula. The nasal minute ventilation (MVₙ) can be approximated by applying a calibration constant (kₙ) to a device minute ventilation (MV_{device}) to convert the device minute ventilation (MV_{device}) to a nasal minute ventilation (MVₙ) signal or measure. In some configurations, the nasal minute ventilation (MVₙ), device minute ventilation (MV_{device}), and the peak inspiratory flow (ûₚₑₐₖ) may be discrete values or a series of discrete values (e.g., a sequence of prior estimates). In some examples, the series of discrete values may begin from the first estimation of the device minute ventilation (MV_{device}), nasal minute ventilation (MVₙ), or peak inspiratory flow (ûₚₑₐₖ) and continue until the end of a device use (therapy) session. Alternatively, a series of discrete values of each of the aforementioned estimates may represent a specific window of time and be continuously overwritten as new values are estimated. For example, a discrete series of values of each of the aforementioned estimates may represent their values over a recent period of time. In some configurations, the period of time can be 15 minutes, 20 minutes, 25 minutes, 30 minutes, 35 minutes, 40 minutes, 45 minutes, 50 minutes, 55 minutes, 60 minutes, 65 minutes, 70 minutes, between 15-20 minutes, between 20-25 minutes, between 25-30 minutes, between 30-35 minutes, between 35-40 minutes, between 40-45 minutes, between 45-50 minutes, between 50-55 minutes, between 55-60 minutes, between 60-65 minutes, between 65-70 minutes, between 15-30 minutes, between 30-45 minutes, between 45-60 minutes, and any values in between those ranges listed, including endpoints.

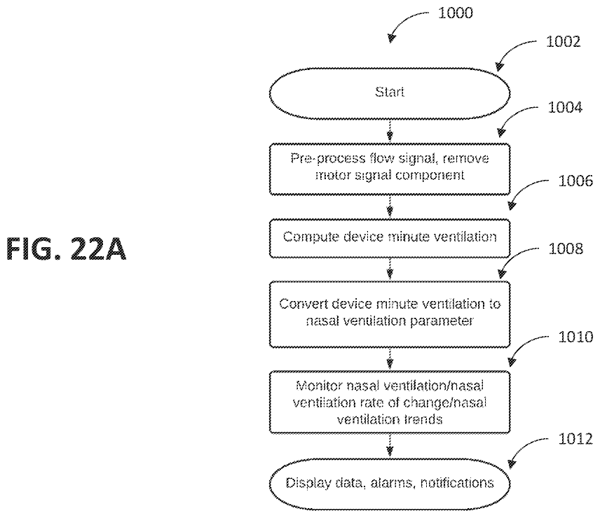

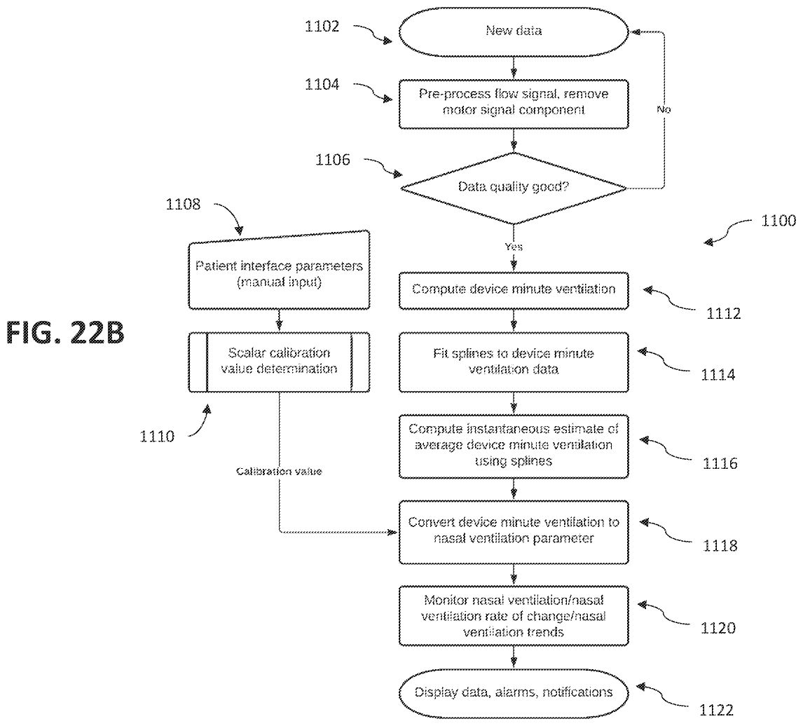

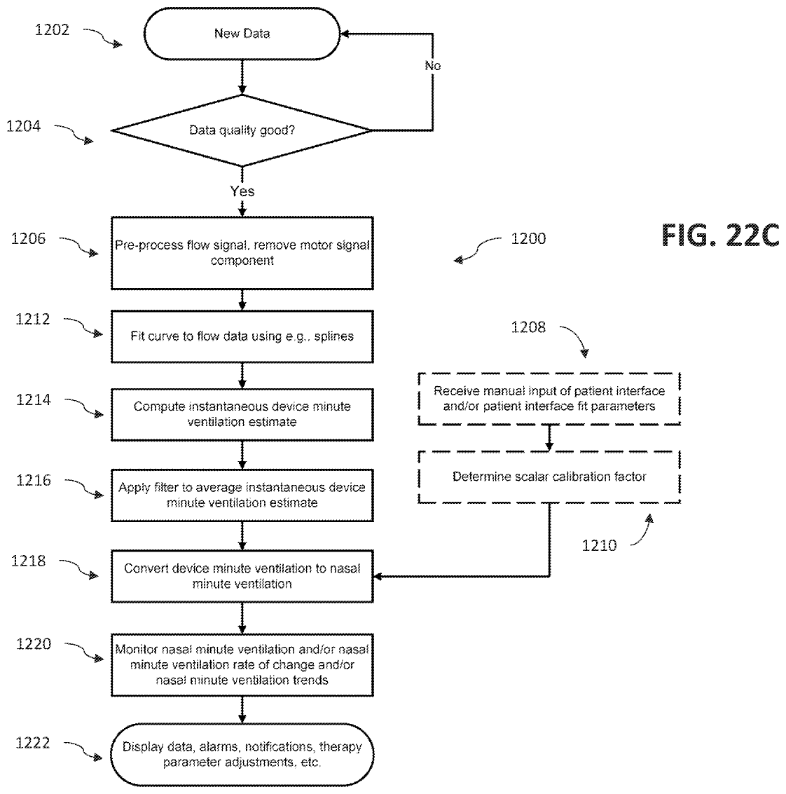

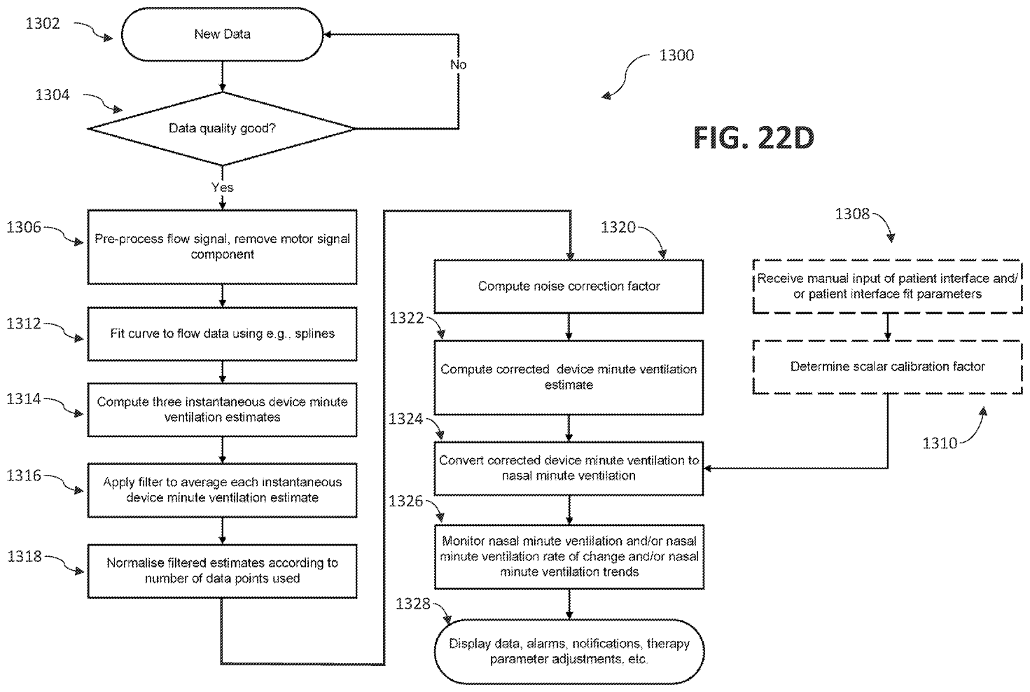

FIGS. 22A, 22B, 22C, and 22D illustrate four flow charts for a method of estimating nasal minute ventilation. FIG. 22A illustrates a simplified method of estimating nasal minute ventilation 1000. FIGS. 22B, 22C, and 22D illustrate a more detailed method of estimating nasal minute ventilation 1100. As will be discussed below, in order to determine the nasal minute ventilation (MVₙ), an estimation of the device minute ventilation (MV_{device}) must first be computed.

As shown in FIG. 22A, a process can be applied to the device minute ventilation (MV_{device}) to obtain the nasal minute ventilation (MVₙ) and use the nasal minute ventilation (MVₙ) to monitor and determine the efficacy of the therapy parameters. The method of estimating nasal minute ventilation 1000 starts by obtaining raw flow data at step 1002. The raw flow data can be acquired from a flow rate sensor such as an ultrasonic flow sensor. The method of estimating nasal minute ventilation 1000 can include pre-processing the raw flow data to remove unwanted signal components at step 1004. Removal of unwanted signal components is described in more detail above. The unwanted signal components can be present from the flow generator motor. In some configurations, unwanted signal components can be generated from other sources (e.g., noise), the unwanted signal components can be primarily from the flow generator motor. For example, the pre-processed flow data can be a flow rate signal comprising components associated with a patient's breathing activity. For example, components associated with the patient's breathing activity may be included in the flow rate signal as changes in the flow rate magnitude (e.g., as fluctuations). Once pre-processed, the flow data can be representative of patient breathing data. The method 1000 can include step 1006 where, assuming the data is of sufficient quality, the pre-processed data can be passed to a device minute ventilation algorithm to calculate the device minute ventilation (MV_{device}). Once the device minute ventilation (MV_{device}) is obtained, step 1008 of the method 1000 can optionally include converting the device minute ventilation (MV_{device}) into the nasal minute ventilation (MVₙ). The nasal minute ventilation (MVₙ) can be estimated by converting and/or transforming the device minute ventilation (MV_{device}) using a scalar calibration constant. As will be discussed in more detail below, the scalar calibration constant can be determined using manually input parameters for the nasal cannula and current flow rate. Once an estimation of the nasal minute ventilation (MVₙ) is calculated, the method 1000 can monitor the patient's nasal minute ventilation, the nasal minute ventilation rate of change, and/or the nasal minute ventilation trends at step 1010. The information regarding the patient's nasal minute ventilation, the nasal minute ventilation rate of change, and/or the nasal minute ventilation trends can be used to provide the user and/or health care professional with information at step 1012. The information associated with the nasal minute ventilation (MVₙ) can be in the form of data displayed, alarms, and/or notification. As discussed above, this can allow the user and/or health care professional to adjust the therapy parameters to improve therapy outcomes.

FIG. 22B illustrates a more detailed flowchart of the method of estimating nasal minute ventilation 1100. As with the method 1000, the method 1100 starts by obtaining raw flow data at step 1102. The raw flow data can be acquired from a flow rate sensor such as an ultrasonic flow sensor. The method 1100 can include pre-processing the raw flow data to remove unwanted signal components at step 1104. Removal of unwanted signal components is described in more detail above. The unwanted signal components can be present from the flow generator motor. Once pre-processed, the flow data can be representative of patient breathing data. The processed flow data can then analyzed at step 1106 to determine whether the data quality is sufficiently good. If not, the flow data is discarded and the method 1100 returns to step 1102 and waits to receive raw flow data.

However, if the data is determined to be of sufficient quality, the data can progress to step 1112 wherein the method 1100 computes the device minute ventilation (MV_{device}) using the processed data. The data can be considered of sufficient quality if it does not include large transient peaks (perhaps due to interface adjustment). The device minute ventilation (MV_{device}) measures the average volume of air being pushed in and out of the device per minute. As shown in step 112, the process for computing the device minute ventilation (MV_{device}) can be done by first fitting splines to the flow data using the least squares criterion. The flow data may be, for example, the most recent pre-filtered flow rate data points. In some configurations, a least squares criterion first approximates the pre-processed flow data (e.g., the breathing signal) and then integrates along the splines to estimate ventilation volumes.

The method 1100 can include step 1116 wherein an instantaneous estimate of device minute ventilation (MV_{device}) is computed using splines. In some examples, the use of splines can be useful over alternative methods (e.g., a series of filters configured to generate statistical measures of the data) because it can perform better (i.e., more accurately fit/interpolate the data) across a wider range of sampling frequencies. This is described in more detail below. The method 1000 can include three different methods of computing instantaneous estimates of the device minute ventilation (MV_{device}). An estimate of the device minute ventilation (MV_{device}) represents the integral of the absolute value of the first term of the line fitted to the data (û_{fit}) of the parameter of the flow of gases, divided by the time range covered by the selected filtered flow rate data points (i.e., a zero-order spline). An estimate of the device minute ventilation (MV_{device}) is represented by the integral of the absolute value of the line fitted to the data (û_{fit}) of the parameter of the flow of gases, divided by the time range covered by the selected filtered flow rate data points (i.e., a first-order spline). To calculate the instantaneous estimate of device minute ventilation (MV_{device}), the estimate can be taken over 1 second or 20 estimates can be taken over 1 second (i.e., a sampling frequency of 20 Hz). The time period for calculating an estimate can be any one of a range of between at least 1-120 seconds, 1-60 seconds, 60-120 seconds, 1-10 seconds, 10-20 seconds, 20-30 seconds, 30-40 seconds, 40-50 seconds, 50-60 seconds, 60-70 seconds, 70-80 seconds, 80-90 seconds, 90-100 seconds, 100-110 seconds, 110-120 seconds, or at least one of 1 second, 10 seconds, 20 seconds, 30 second, 40 seconds, 50 seconds, 60 seconds, 70 seconds, 80 seconds, 90 seconds, 100 seconds, 110 seconds, and 120 seconds.

An estimate of the device minute ventilation (MV_{device}) represents the average of the absolute value of the curve fitted to the data of the parameter of the flow of gases (i.e., computed without using splines for data interpolation). Any of the aforementioned estimates can be used in the method 1100 as the input, although each have advantages and disadvantages depending on the random error in the sensor data and the patient respiration rate. The method 1100 can use an estimate of the device minute ventilation (MV_{device}) that represents the integral of the absolute value of the first term of the line fitted to the signal. This estimate can be the most resilient to and least influenced by random error while also being the most influenced by respiration rate.

Once the method 1100 obtains an estimate of the device minute ventilation (MV_{device}) at step 1114, the device minute ventilation (MV_{device}) can optionally be converted to a nasal minute ventilation (MVₙ) estimate at step 1118. The device minute ventilation (MV_{device}) can optionally be converted to the nasal minute ventilation (MVₙ) using a scalar calibration constant (kₙ). As shown in step 1108 and step 1110, the calibration constant (kₙ) can, optionally, be at least in part calculated using manually-input patient interface parameters at step 1108 and then determining the scalar calibration value at step 1110. The patient interface parameters that are manually input can be related to the nasal cannula and the current flow rate. These parameters can include, but are not limited to, the cannula type, patient size, nostril occlusion (or an estimation thereof), and naris diameter. These parameters can be estimates of the aforementioned cannula type, patient size, and naris diameter. Alternatively, these parameters can be automatically determined by the device, by, for example, using an automatic peripheral component detection system or receiving flow data from a flow rate sensor. The calibration constant (kₙ) may be computed via a calibration phase or process wherein a full sealing face mask with an outlet flow sensor is temporarily placed over the nasal cannula on the patient. Using the full sealing face mask, various flow parameters can be captured and input to the calibration constant determination function, algorithm, or method. Alternatively, the calibration constant may be derived from an estimate of the flow conductance that is calculated in the controller.

Once an estimation of the nasal minute ventilation (MVₙ) is calculated, the method 1100 can monitor the patients' nasal minute ventilation, the nasal minute ventilation rate of change, and/or the nasal minute ventilation trends at step 1120. The information regarding the patient's nasal minute ventilation, the nasal minute ventilation rate of change, and/or the nasal minute ventilation trends can be used to provide the user and/or health care professional with information at step 1122. As discussed above, the information associated with the nasal minute ventilation (MVₙ) can be in the form of data displayed, alarms, and/or notification. By monitoring either nasal minute ventilation (MVₙ) itself, the rate of the change of nasal minute ventilation (MVₙ), or the longer-term trends in nasal minute ventilation (MVₙ), the device controller may be configured to present audio, visual, or audio-visual alarms or notifications for the user and/or health care professional. These alarms can include prompts to adjust therapy parameters such as flow rate. The alarms can be configured to trigger based on suggested threshold or manually input thresholds. As discussed above, this can allow the user and/or health care professional to adjust the therapy parameters to improve therapy outcomes. The trend in nasal minute ventilation (MVₙ) can also be displayed on a device screen as a plot, chart, or graph. Based on the illustrated trend data, notifications and/or alarms can be triggered based on them.

FIG. 22C illustrates another more detailed flowchart of the method of estimating nasal minute ventilation 1200. As with the method 1000 and method 1100, the method 1200 starts by obtaining raw flow data at step 1202. The raw flow data can be acquired from a flow rate sensor such as an ultrasonic flow sensor. In some configurations, at step 1204, the raw flow data is first analyzed to determine whether the data quality is sufficiently good. If the raw flow data is determined of sufficient quality, the data can be pre-processed to remove unwanted signal components. If the raw flow data is of insufficient quality, the flow data is discarded and the method 1200 returns to step 1202 and waits to receive additional raw flow data. In some examples, the method 1200 can include pre-processing the raw flow data to remove unwanted signal components at step 1206. Removal of unwanted signal components is described in more detail above. The unwanted signal components can be present from the flow generator motor. Once pre-processed, the flow data can be representative of patient breathing data. In some configurations, once the flow data is pre-processed, the data can progress to step 1212 wherein the method 1200 fits a curve to the flow data. The data can be considered of sufficient quality if it does not include large transient peaks (perhaps due to interface adjustment). The device minute ventilation (MV_{device}) measures the average volume of air being pushed in and out of the device per minute. The process for fitting a curve to the flow data can be done by first fitting splines to the flow data using a least squares criterion. In some configurations, the fitted line can be represented (approximately) by:

$${\hat{u}}_{fit} = m + {st}^{*}$$

In the equation provided above, m is a fit parameter corresponding to the mean of flow data, s is the slope (i.e., gradient), and t* is a linear range of normalized time parameters. In some configurations t* is a linear range of normalized time parameters wherein the “oldest” time point in the flow data is equal to −1 and the most “recent” time point in the flow data is equal to 1.

In some configurations, other methods of function approximation may be used as well. The flow data may be, for example, the most recent pre-filtered flow rate data points. In some examples, a least squares method can be used to approximate the pre-processed flow data (e.g., the breathing signal) and then integrates along the splines to estimate ventilation volumes. In some configurations, the controller can perform a variety of line and/or curve fitting techniques to fit the one or more functions to the selected portion of the flow parameter variation data. This can include, for example, the non-limiting example techniques of regression analysis, interpolation, extrapolation, linear least squares, non-linear least squares, total least squares, simple linear regressions, robust simple linear regression, polynomial regression, orthogonal regression, Deming regression, linear segmented regression, regression dilution, and/or others. In some configurations, the one or more functions, which includes at least those above, can generate a curve. In some configurations, the curve can be a line. The lines or curves described herein can include a plurality of curves, vertices, and/or other features. The lines described herein can be straight, angled, and/or horizontal. In some examples, the lines described herein can be a line of best fit.

The method 1200 can include step 1214 wherein an instantaneous estimate of device minute ventilation (MV_{device}) is computed using the data of the curve constructed by the fitted splines. The method 1000 can include three different methods of computing instantaneous estimates of the device minute ventilation (MV_{device}). An estimate of the device minute ventilation (MV_{device}) is represented by the integral of the absolute value of the first term of the line fitted to the data (û_{fit}) of the parameter of the flow of gases, divided by the time range covered by the selected filtered flow rate data points (i.e., a zero-order spline). An estimate of the device minute ventilation (MV_{device}) is represented by the integral of the absolute value of the line fitted to the data (fit) of the parameter of the flow of gases, divided by the time range covered by the selected filtered flow rate data points (i.e., a first-order spline). To calculate the instantaneous estimate of device minute ventilation (MV_{device}), the estimate can be taken over 1 second or 20 estimates can be taken over 1 second (i.e., a sampling frequency of 20 Hz). The time period for calculating an estimate can be any one of a range of between at least 1-120 seconds, 1-60 seconds, 60-120 seconds, 1-10 seconds, 10-20 seconds, 20-30 seconds, 30-40 seconds, 40-50 seconds, 50-60 seconds, 60-70 seconds, 70-80 seconds, 80-90 seconds, 90-100 seconds, 100-110 seconds, 110-120 seconds, or at least one of 1 second, 10 seconds, 20 seconds, 30 second, 40 seconds, 50 seconds, 60 seconds, 70 seconds, 80 seconds, 90 seconds, 100 seconds, 110 seconds, and 120 seconds.

An estimate of the device minute ventilation (MV_{device}) represents the average of the absolute value of the curve fitted to the data of the parameter of the flow of gases (i.e., computed without using splines for data interpolation). Any of the aforementioned estimates can be used in the method 1200 as the input, although each have advantages and disadvantages depending on the random error in the sensor data and the patient respiration rate. The method 1200 can use an estimate of the device minute ventilation (MV_{device}) that represents the integral of the absolute value of the first term of the line fitted to the signal. This estimate can be the most resilient to and least influenced by random error while also being the most influenced by respiration rate. In some implementations, the method 1200 can skip step 1212 and the pre-processed flow data can directly proceed to step 1214 wherein the method 1200 can conduct a direct computation of the average of the selected pre-processed flow rate data points.

Once the method 1200 obtains an estimate of the device minute ventilation (MV_{device}) at step 1214, the device minute ventilation (MV_{device}) can optionally be converted to a nasal minute ventilation (MVₙ) estimate at step 1218. Before the device minute ventilation (MV_{device}) is converted to the estimate of nasal minute ventilation (MVₙ), a filter can be applied to average the instantaneous device minute ventilation (MV_{device}) at step 1216. In some configurations, each estimate or sequence of estimates captured over multiple repetitions of the previously described steps can be averaged or “smoothed” using a filter (e.g., an exponential filter). In some examples, this step can occur after the initial estimation but prior to any additional processing steps (i.e., converting to an estimate of nasal minute ventilation (MVₙ)). In some configurations, the device minute ventilation (MV_{device}) can be converted to the nasal minute ventilation (MVₙ) using a scalar calibration constant (kₙ). As shown in step 1208 and step 1210, the calibration constant (kₙ), optionally, can be at least in part calculated using manually-input patient interface parameters at step 1208 and then determining the scalar calibration value at step 1210. The patient interface parameters that are manually input can be related to the nasal cannula and the current flow rate. These parameters can include, but are not limited to, the cannula type, patient size, nostril occlusion (or an estimation thereof), and naris diameter. These parameters can be estimates of the aforementioned cannula type, patient size, and naris diameter. Alternatively, these parameters can be automatically determined by the device, by, for example, using an automatic peripheral component detection system or receiving flow data from a flow rate sensor. The calibration constant (kₙ) may be computed via a calibration phase or process wherein a full sealing face mask with an outlet flow sensor is temporarily placed over the nasal cannula on the patient. Using the full sealing face mask, various flow parameters can be captured and input to the calibration constant determination function, algorithm, or method. Alternatively, the calibration constant may be derived from an estimate of the flow conductance that is calculated in the controller. The flow conductance may be calculated in the controller based on a determined flow rate of the gases and a determined pressure of the flow rate. Alternatively, several flow conductance values may be stored in a look table wherein the controller is configured to select a flow conductance from the look up table.

Once an estimation of the nasal minute ventilation (MVₙ) is calculated, the method 1200 can monitor the patients' nasal minute ventilation, the nasal minute ventilation rate of change, and/or the nasal minute ventilation trends at step 1220. The information regarding the patient's nasal minute ventilation, the nasal minute ventilation rate of change, and/or the nasal minute ventilation trends can be used to provide the user and/or health care professional with information at step 1222. As discussed above, the information associated with the nasal minute ventilation (MVₙ) can be in the form of data displayed, alarms, and/or notification. By monitoring either nasal minute ventilation (MVₙ) itself, the rate of the change of nasal minute ventilation (MVₙ), or the longer-term trends in nasal minute ventilation (MVₙ), the device controller may be configured to present audio, visual, or audio-visual alarms or notifications for the user and/or health care professional. These alarms can include prompts to adjust therapy parameters such as flow rate. The alarms can be configured to trigger based on suggested threshold or manually input thresholds. As discussed above, this can allow the user and/or health care professional to adjust the therapy parameters to improve therapy outcomes. The trend in nasal minute ventilation (MVₙ) can also be displayed on a device screen as a plot, chart, or graph. Based on the illustrated trend data, notifications and/or alarms can be triggered based on them.

In some configurations, an estimate of device minute ventilation (MV_{device}) is determined, filtered, and then converted into a nasal minute ventilation (MVₙ) by taking an estimate that involves taking the integral of the absolute value of the first term of the line fitted to the flow rate signal (e.g., zero-order spline). In some examples, the device minute ventilation (MV_{device}) is estimated for each of the data points using all three approaches discussed above. As previously mentioned, the three methods include: (1) where an estimate of the device minute ventilation (MV_{device}) is represented by the integral of the absolute value of the first term of the line fitted to the data (fit) of the parameter of the flow of gases, divided by the time range covered by the selected filtered flow rate data points (i.e., a zero-order spline); (2) where an estimate of the device minute ventilation (MV_{device}) is represented by the integral of the absolute value of the line fitted to the data (û_{fit}) of the parameter of the flow of gases, divided by the time range covered by the selected filtered flow rate data points (i.e., a first-order spline); and (3) where an estimate of the device minute ventilation (MV_{device}) represents the average of the absolute value of the curve fitted to the data of the parameter of the flow of gases (i.e., computed without using splines for data interpolation).

FIG. 22D illustrates another flowchart of the method of estimating nasal minute ventilation 1300 wherein the device minute ventilation (MV_{device}) is estimated for each of the data points using all three approaches discussed above. As with the method 1000, 1100, 1200, the method 1300 starts by obtaining raw flow data at step 1302. The raw flow data can be acquired from a flow rate sensor such as an ultrasonic flow sensor. In some configurations, at step 1304, the raw flow data is first analyzed to determine whether the data quality is sufficiently good. If the raw flow data is determined to be of sufficient quality, the data can be pre-processed to remove unwanted signal components. If the raw flow data is of insufficient quality, the flow data is discarded and the method 1300 returns to step 1302 and waits to receive additional raw flow data. In some examples, the method 1300 can include pre-processing the raw flow data to remove unwanted signal components at step 1306. Removal of unwanted signal components is described in more detail above. The unwanted signal components can be present from the flow generator motor. Once pre-processed, the flow data can be representative of patient breathing data. In some configurations, once the flow data is pre-processed, the data can progress to step 1312 wherein the method 1300 fits a curve to the flow data. The data can be considered of sufficient quality if it does not include large transient peaks (perhaps due to interface adjustment). The device minute ventilation (MV_{device}) measures the average volume of air being pushed in and out of the device per minute. The process for fitting a curve to the flow data can be done by first fitting splines to the flow data using a least squares criterion. In some configurations, the fitted line can be represented (approximately) by:

$${\hat{u}}_{fit} = m + {st}^{*}$$

In the equation provided above, m is a fit parameter corresponding to the mean of flow data, s is the slope (i.e., gradient), and t* is a linear range of normalized time parameters. In some configurations t* is a linear range of normalized time parameters wherein the “oldest” time point in the flow data is equal to −1 and the most “recent” time point in the flow data is equal to 1.

In some configurations, other methods of function approximation may be used as well. The flow data may be, for example, the most recent pre-filtered flow rate data points. In some examples, a least squares method can be used to approximate the pre-processed flow data (e.g., the breathing signal) and then integrates along the splines to estimate ventilation volumes. In some configurations, the controller can perform a variety of line and/or curve fitting techniques to fit the one or more functions to the selected portion of the flow parameter variation data. This can include, for example, the non-limiting example techniques of regression analysis, interpolation, extrapolation, linear least squares, non-linear least squares, total least squares, simple linear regressions, robust simple linear regression, polynomial regression, orthogonal regression, Deming regression, linear segmented regression, regression dilution, and/or others. In some configurations, the one or more functions, which includes at least those above, can generate a curve. In some configurations, the curve can be a line. The lines or curves described herein can include a plurality of curves, vertices, and/or other features. The lines described herein can be straight, angled, and/or horizontal. In some examples, the lines described herein can be a line of best fit.

The method 1300 can include step 1314 wherein three instantaneous estimates of device minute ventilation (MV_{device}) are computed using the data of the curve constructed by the fitted splines. The method 1300 can include three different methods of computing instantaneous estimates of the device minute ventilation (MV_{device}). In some configurations, one of the three estimates is an estimate of the device minute ventilation (MV_{device}) represented by the integral of the absolute value of the first term of the line fitted to the data (ûfit) of the parameter of the flow of gases, divided by the time range covered by the selected filtered flow rate data points (i.e., a zero-order spline). In some examples, another of the three estimates is an estimate of the device minute ventilation (MV_{device}) represented by the integral of the absolute value of the line fitted to the data (û_{fit}) of the parameter of the flow of gases, divided by the time range covered by the selected filtered flow rate data points (i.e., a first-order spline). To calculate the instantaneous estimate of device minute ventilation (MV_{device}), the estimate can be taken over 1 second or 20 estimates can be taken over 1 second (i.e., a sampling frequency of 20 Hz). The time period for calculating an estimate can be any one of a range of between at least 1-120 seconds, 1-60 seconds, 60-120 seconds, 1-10 seconds, 10-20 seconds, 20-30 seconds, 30-40 seconds, 40-50 seconds, 50-60 seconds, 60-70 seconds, 70-80 seconds, 80-90 seconds, 90-100 seconds, 100-110 seconds, 110-120 seconds, or at least one of 1 second, 10 seconds, 20 seconds, 30 second, 40 seconds, 50 seconds, 60 seconds, 70 seconds, 80 seconds, 90 seconds, 100 seconds, 110 seconds, and 120 seconds. In some configurations, another of the three estimates is an estimate of the device minute ventilation (MV_{device}) represented by the average of the absolute value of the curve fitted to the data of the parameter of the flow of gases (i.e., computed without using splines for data interpolation). In some configurations, the first MV_{device} estimate (i.e., zero-order spline) is most resilient to noise/least dependent upon it, and is most influenced by patient respiratory rate. In some examples, the second MV_{device} estimate (i.e., first-order spline) is very noise-dependent but is less dependent of respiratory rate. In some configurations, the third MV_{device} estimate (i.e., wherein there are no splines/interpolation and the estimate is a direct averaging of a series of instantaneous absolute values) is highly affected by noise and is independent of respiratory rate. It is further noted that all three estimates are flow-variant, meaning they will vary according to the blower flow output.

In some configurations, the method 1300 can skip step 1312 and the pre-processed flow data can directly proceed to step 1314 wherein the method 1300 can conduct a direct computation of the average of the selected pre-processed flow rate data points.

Once the method 1300 obtains the three estimates of the device minute ventilation (MV_{device}) at step 1314, a filter can be applied to the device minute ventilation (MV_{device}) to average the instantaneous device minute ventilation (MV_{device}) at step 1316. In some configurations, each estimate or sequence of estimates captured over multiple repetitions of the previously described steps can be averaged or “smoothed” using a filter (e.g., an exponential filter). In some examples, this step can occur after the initial estimation but prior to any additional processing steps (i.e., converting to an estimate of nasal minute ventilation (MVₙ)).

In the method 1300, there are three measurements (i.e., the first MV_{device} estimate, the second MV_{device} estimate, and the third MV_{device} estimate) and three unknown values or signal components that can constitute and/or contribute to the device minute ventilation signal estimated. These unknown values and signal components can include, for example, noise, respiratory rate (e.g., the speed of flow change induced by the patient), and the underlying device minute ventilation signal. Therefore, there exists a set of three analytic expressions or equations that may be solved simultaneously to derive expressions for noise, respiratory rate, and device minute ventilation. In some configurations, simultaneously solving the three analytic expressions or equations can be highly computationally expensive and therefore demanding on processing hardware in embedded device applications, such as in medical devices. In some configurations, in the method 1300, the algorithm of the method 1300 first proceeds to step 1318 to normalize the three MV_{device} estimates according to the number of data points used in each of the estimations. In some examples, the method 1300 can include step 1320 wherein a noise correction factor can be computed, wherein the noise correction factor has a relationship with the signal-noise ratio. The calculation of a noise correction factor in step 1320 can be similar to any of those disclosed in Applicant's application number PCT/IB2020/051816, filed Mar. 4, 2020, entitled PATIENT ATTACHMENT DETECTION IN RESPIRATORY FLOW THERAPY SYSTEMS, the entirety of which is incorporated by reference herein. In some configurations, a noise correction factor that is related to one or more of the normalized MV_{device} estimates may be computed.

In some configurations, the method 1300 can include step 1322 wherein the algorithm can use a pre-defined fitted curve that relates the normalized minute ventilation estimates and the noise correction factor to one of the device minute ventilation (MV_{device}) estimates. In some configurations, the fitting function may comprise, at least partially, some numerically-derived terms. In some configurations, this corrected curve can approximate the output of an analytic expression for the device minute ventilation (MV_{device}). This can provide a device minute ventilation (MV_{device}) that has a minimal noise and respiratory rate dependency.

In some configurations, the method 1300 can include step 1324 wherein the device minute ventilation (MV_{device}) can be converted to an estimate of nasal minute ventilation (MVₙ). In some examples, each estimate or sequence of estimates captured over multiple repetitions of the previously described steps can be averaged or “smoothed” using a filter (e.g., an exponential filter). In some examples, this step can occur after the initial estimation but prior to any additional processing steps (i.e., converting to an estimate of nasal minute ventilation (MVₙ)). In some configurations, the device minute ventilation (MV_{device}) can be converted to the nasal minute ventilation (MVₙ) using a scalar calibration constant (kₙ). As shown in step 1308 and step 1310, the calibration constant (kₙ), optionally, can be at least in part calculated using manually-input patient interface parameters at step 1308 and then determining the scalar calibration value at step 1310. The patient interface parameters that are manually input can be related to the nasal cannula and the current flow rate. These parameters can include, but are not limited to, the cannula type, patient size, nostril occlusion (or an estimation thereof), and naris diameter. These parameters can be estimates of the aforementioned cannula type, patient size, and naris diameter. Alternatively, these parameters can be automatically determined by the device, by, for example, using an automatic peripheral component detection system or receiving flow data from a flow rate sensor. The calibration constant (kₙ) may be computed via a calibration phase or process wherein a full sealing face mask with an outlet flow sensor is temporarily placed over the nasal cannula on the patient. Using the full sealing face mask, various flow parameters can be captured and input to the calibration constant determination function, algorithm, or method. Alternatively, the calibration constant may be derived from an estimate of the flow conductance that is calculated in the controller. The flow conductance may be determined as described earlier.

Once an estimation of the nasal minute ventilation (MVₙ) is calculated, the method 1300 can monitor the patients' nasal minute ventilation, the nasal minute ventilation rate of change, and/or the nasal minute ventilation trends at step 1326. The information regarding the patient's nasal minute ventilation, the nasal minute ventilation rate of change, and/or the nasal minute ventilation trends can be used to provide the user and/or health care professional with information at step 1328. As discussed above, the information associated with the nasal minute ventilation (MVₙ) can be in the form of data displayed, alarms, and/or notification. By monitoring either nasal minute ventilation (MVₙ) itself, the rate of the change of nasal minute ventilation (MVₙ), or the longer-term trends in nasal minute ventilation (MVₙ), the device controller may be configured to present audio, visual, or audio-visual alarms or notifications for the user and/or health care professional. These alarms can include prompts to adjust therapy parameters such as flow rate. The alarms can be configured to trigger based on suggested threshold or manually input thresholds. As discussed above, this can allow the user and/or health care professional to adjust the therapy parameters to improve therapy outcomes. The trend in nasal minute ventilation (MVₙ) can also be displayed on a device screen as a plot, chart, or graph. Based on the illustrated trend data, notifications and/or alarms can be triggered based on them.

FIG. 23 illustrates a flowchart of method of estimating a normalized device minute ventilation 1400. Unlike the methods illustrated in FIGS. 22B-22D, instead of computing a nasal minute ventilation, the respiratory device takes the corrected device minute ventilation and normalizes using the device flow rate. In some configurations, this can render an estimate of device minute ventilation that is flow-invariant and independent of the device flow rate. In some examples, the estimated device minute ventilation is also independent of nasal cannula fit and can provide a versatile indicator of patient minute ventilation without converting the initially estimated device minute ventilation to nasal minute ventilation.

The method 1400 starts by obtaining raw flow data at step 1402. The raw flow data can be acquired from a flow rate sensor such as an ultrasonic flow sensor. In some configurations, at step 1404, the raw flow data is first analyzed to determine whether the data quality is sufficiently good. If the raw flow data is determined to be of sufficient quality, the data can be pre-processed to remove unwanted signal components. If the raw flow data is of insufficient quality, the flow data is discarded and the method 1400 returns to step 1402 and waits to receive additional raw flow data. In some examples, the method 1400 can include pre-processing the raw flow data to remove unwanted signal components at step 1406. Removal of unwanted signal components is described in more detail above. The unwanted signal components can be present from the flow generator motor.

In some configurations, once the flow data is pre-processed, the data can progress to step 1408 wherein the method 1400 fits a curve to the flow data. The data can be considered of sufficient quality if it does not include large transient peaks (perhaps due to interface adjustment). The device minute ventilation (MV_{device}) measures the average volume of air being pushed in and out of the device per minute. The process for fitting a curve to the flow data can be done by first fitting splines to the flow data using a least squares criterion. In some configurations, the fitted line can be represented (approximately) by:

$${\hat{u}}_{fit} = m + {st}^{*}$$

In the equation provided above, m is a fit parameter corresponding to the mean of flow data, s is the slope (i.e., gradient), and t* is a linear range of normalized time parameters. In some configurations t* is a linear range of normalized time parameters wherein the “oldest” time point in the flow data is equal to −1 and the most “recent” time point in the flow data is equal to 1.

In some configurations, other methods of function approximation may be used as well. The flow data may be, for example, the most recent pre-filtered flow rate data points. In some examples, a least squares method can be used to approximate the pre-processed flow data (e.g., the breathing signal) and then integrates along the splines to estimate ventilation volumes. In some configurations, the controller can perform a variety of line and/or curve fitting techniques to fit the one or more functions to the selected portion of the flow parameter variation data. This can include, for example, the non-limiting example techniques of regression analysis, interpolation, extrapolation, linear least squares, non-linear least squares, total least squares, simple linear regressions, robust simple linear regression, polynomial regression, orthogonal regression, Deming regression, linear segmented regression, regression dilution, and/or others. In some configurations, the one or more functions, which includes at least those above, can generate a curve. In some configurations, the curve can be a line. The lines or curves described herein can include a plurality of curves, vertices, and/or other features. The lines described herein can be straight, angled, and/or horizontal. In some examples, the lines described herein can be a line of best fit.

The method 1400 can include step 1410 wherein three instantaneous estimates of device minute ventilation (MV_{device}) are computed using the data of the curve constructed by the fitted splines. The method 1400 can include three different methods of computing instantaneous estimates of the device minute ventilation (MV_{device}). In some configurations, one of the three estimates is an estimate of the device minute ventilation (MV_{device}) represented by the integral of the absolute value of the first term of the line fitted to the data (u fit) of the parameter of the flow of gases, divided by the time range covered by the selected filtered flow rate data points (i.e., a zero-order spline). In some examples, another of the three estimates is an estimate of the device minute ventilation (MV_{device}) represented by the integral of the absolute value of the line fitted to the data (û_{fit}) of the parameter of the flow of gases, divided by the time range covered by the selected filtered flow rate data points (i.e., a first-order spline). To calculate the instantaneous estimate of device minute ventilation (MV_{device}), the estimate can be taken over 1 second or 20 estimates can be taken over 1 second (i.e., a sampling frequency of 20 Hz). The time period for calculating an estimate can be any one of a range of between at least 1-120 seconds, 1-60 seconds, 60-120 seconds, 1-10 seconds, 10-20 seconds, 20-30 seconds, 30-40 seconds, 40-50 seconds, 50-60 seconds, 60-70 seconds, 70-80 seconds, 80-90 seconds, 90-100 seconds, 100-110 seconds, 110-120 seconds, or at least one of 1 second, 10 seconds, 20 seconds, 30 second, 40 seconds, 50 seconds, 60 seconds, 70 seconds, 80 seconds, 90 seconds, 100 seconds, 110 seconds, and 120 seconds. In some configurations, another of the three estimates is an estimate of the device minute ventilation (MV_{device}) represented by the average of the absolute value of the curve fitted to the data of the parameter of the flow of gases (i.e., computed without using splines for data interpolation). In some configurations, the first MV_{device} estimate (i.e., zero-order spline) is most resilient to noise/least dependent upon it, and is most influenced by patient respiratory rate. In some examples, the second MV_{device} estimate (i.e., first-order spline) is very noise-dependent but is less dependent of respiratory rate. In some configurations, the third MV_{device} estimate (i.e., wherein there are no splines/interpolation and the estimate is a direct averaging of a series of instantaneous absolute values) is highly affected by noise and is independent of respiratory rate. It is further noted that all three estimates are flow-variant, meaning they will vary according to the blower flow output.

In some configurations, the method 1400 can skip step 1408 and the pre-processed flow data can directly proceed to step 1410 wherein the method 1300 can conduct a direct computation of the average of the selected pre-processed flow rate data points.

Once the method 1400 obtains the three estimates of the device minute ventilation (MV_{device}) at step 1410, a filter can be applied to the device minute ventilation (MV_{device}) to average the instantaneous device minute ventilation (MV_{device}) at step 1412. In some configurations, each estimate or sequence of estimates captured over multiple repetitions of the previously described steps can be averaged or “smoothed” using a filter (e.g., an exponential filter). In some examples, this step can occur after the initial estimation but prior to any additional processing steps (i.e., converting to an estimate of nasal minute ventilation (MVₙ)).

In the method 1400, there are three measurements (i.e., the first MV_{device} estimate, the second MV_{device} estimate, and the third MV_{device} estimate) and three unknown values or signal components that can constitute and/or contribute to the device minute ventilation signal estimated. These unknown values and signal components can include, for example, noise, respiratory rate (e.g., the speed of flow change induced by the patient), and the underlying device minute ventilation signal. Therefore, there exists a set of three analytic expressions or equations that may be solved simultaneously to derive expressions for noise, respiratory rate, and device minute ventilation. In some configurations, simultaneously solving the three analytic expressions or equations can be highly computationally expensive and therefore demanding on processing hardware in embedded device applications, such as in medical devices. In some configurations, in the method 1400, the algorithm of the method 1400 first proceeds to step 1414 to normalize the three MV_{device} estimates according to the number of data points used in each of the estimations. In some examples, the method 1400 can include step 1416 wherein a noise correction factor can be computed, wherein the noise correction factor has a relationship with the signal-noise ratio. The calculation of a noise correction factor in step 1416 can be similar to any of those disclosed in Applicant's application number PCT/IB2020/051816, filed Mar. 4, 2020, entitled PATIENT ATTACHMENT DETECTION IN RESPIRATORY FLOW THERAPY SYSTEMS, the entirety of which is incorporated by reference herein. In some configurations, a noise correction factor that is related to one or more of the normalized MV_{device} estimates may be computed.

In some configurations, the method 1400 can include step 1418 wherein the algorithm can use a pre-defined fitted curve that relates the normalized minute ventilation estimates and the noise correction factor to one of the device minute ventilation (MV_{device}) estimates. In some configurations, the fitting function may comprise, at least partially, some numerically-derived terms. In some configurations, this corrected curve can approximate the output of an analytic expression for the corrected device minute ventilation (MV_{device}). This can provide a corrected device minute ventilation (MV_{device}) that has a minimal noise and respiratory rate dependency.

In some configurations, the method 1400 can include 1420 wherein the corrected device minute ventilation (MV_{device}) is normalized using the device flow rate. In some examples, the same data that was previously used in the prior steps (i.e., good-quality, pre-processed flow rate data) is used to reach the corrected device minute ventilation (MV_{device}) and provide an estimate of device minute ventilation that is flow-invariant and independent of the device flow rate. As mentioned previously, the corrected device minute ventilation (MV_{device}) can be independent of nasal cannula fit and provides a versatile indicator of patient minute ventilation (even without converting to nasal minute ventilation).

Once an estimation of the normalized device minute ventilation (MV_{device}) is calculated, the method 1400 can monitor the patients' nasal minute ventilation, the nasal minute ventilation rate of change, and/or the nasal minute ventilation trends at step 1422. The information regarding the patient's nasal minute ventilation, the nasal minute ventilation rate of change, and/or the nasal minute ventilation trends can be used to provide the user and/or health care professional with information at step 1424. As discussed above, the information associated with the normalized device minute ventilation (MV_{device}) can be in the form of data displayed, alarms, and/or notification. By monitoring either normalized device minute ventilation (MV_{device}) itself, the rate of the change of the normalized device minute ventilation (MV_{device}) or the longer-term trends in normalized device minute ventilation (MV_{device}), the device controller may be configured to present audio, visual, or audio-visual alarms or notifications for the user and/or health care professional. These alarms can include prompts to adjust therapy parameters such as flow rate. The alarms can be configured to trigger based on suggested threshold or manually input thresholds. As discussed above, this can allow the user and/or health care professional to adjust the therapy parameters to improve therapy outcomes. The trend in the normalized device minute ventilation (MV_{device}) can also be displayed on a device screen as a plot, chart, or graph. Based on the illustrated trend data, notifications and/or alarms can be triggered based on them. In some examples, the patient's nasal minute ventilation can be monitored indirectly by monitoring the normalized device minute ventilation.

A user and/or health care professional can monitor the rate of change of the nasal minute ventilation (MVₙ) as opposed to its average or instantaneous value. The determination of the calibration constant (kₙ) can be difficult and potentially inaccurate and unreliable. In certain instances, direct usage of the immediate value of the nasal minute ventilation (MVₙ) may have limited benefit because of the issue of the accuracy and/or reliability of the immediate value. In some configurations, the accuracy and/or reliability of the immediate value of the nasal minute ventilation (MVₙ) may be limited because of the accuracy and/or reliability of the calibration constant (kₙ). However, while the value of the nasal minute ventilation (MVₙ) in certain examples may have significant error, the trend in the nasal minute ventilation (MVₙ) is accurately or reliably representative of patient breathing effort over time. For example, a decreasing or increasing trend of the nasal minute ventilation (MVₙ) can indicate the efficacy of therapy or the worsening of a patient's health. Due to the subjectivity of the nasal minute ventilation (MVₙ), changes or trends of the nasal minute ventilation (MVₙ) can be more useful to users and health care professionals as opposed to the immediate value itself. The nasal minute ventilation (MVₙ) can be subjective because each patient's nasal minute ventilation (MVₙ) readings might different as a result of each patient's individual height, mass, etc. With significant variation between patients, it may be difficult for a clinician to draw any strong conclusions based solely of a single nasal minute ventilation (MVₙ) value. For example, if the nasal minute ventilation (MVₙ) increases over time, this can be an indication to the user and/or health care professional that the therapy parameters are effective as the patient's lungs are strengthening over time. However, if the nasal minute ventilation (MVₙ) decreases over time, this can indicate to the user or healthcare professional that the patient's lung function is deteriorating. For example, a decrease in the nasal minute ventilation (MVₙ) can indicate worsening asthma symptoms, an increase in scar tissue, reduced patient energy, increased bacterial or viral infection, and increased fluid in the lungs. The trend information of the nasal minute ventilation (MVₙ) can be particularly useful when the nasal high flow system is used in a home setting. The trend information can help identify patient deterioration without the need for user know-how in interpreting individual data points and values.

If the respiratory rate RR is known, an estimate of patient tidal volume (V_{T}) may be computed using the nasal minute ventilation (MVₙ):

$${V}_{T} = \frac{{MV}_{n}}{RR} .$$

The value of the tidal volume (V_{T}) can be useful as another breathing effort or health indicator particularly when a patient's overall lung dead space is known. As tidal volume (V_{T}) is a well-understood and common metric used by clinicians and other healthcare professionals, it can be helpful to provide tidal volume (V_{T}) information or alarms, in addition or alternatively to nasal minute ventilation (MVₙ). The tidal volume estimate may be displayed on the display of the respiratory therapy apparatus. The estimated tidal volume may also be transmitted to a remote server or a remote patient management system for incorporation into a patient report. In one configuration the respiratory apparatus may also raise an alarm (e.g. an audible and visual alarm) if the estimated tidal volume drops below a threshold. There may also be an alarm if the estimated tidal volume is above a threshold. In one example a clinician may be able to set a minimum tidal volume threshold via a user interface (e.g. touchscreen or combination of touchscreen and buttons) of the respiratory apparatus. The controller may be configured to repeatedly estimate tidal volume as described above and compare with the clinician set threshold. If the tidal volume is below the set threshold then an alarm may be raised to indicate to a clinician that the patient tidal volume is not adequate. Optionally an upper threshold may also be set and an alarm may be provided if the tidal volume is above the upper threshold.

### Peak Inspiratory Flow

The controller can include processes that are configured to calculate an estimate of peak inspiratory flow (ûₚₑₐₖ). An estimation of the peak inspiratory flow (ûₚₑₐₖ) is a metric of patient peak inspiratory flow. An estimation of the peak inspiratory flow (ûₚₑₐₖ) can be used to estimate patient breathing demands and effort.

The peak inspiratory flow (ûₚₑₐₖ) can be estimated by converting and/or transforming the nasal minute ventilation (MVₙ) using another scalar calibration constant. This can be done by simply applying a real number as a scaling factor to the nasal minute ventilation (MVₙ) estimate. Similar to the method for estimating the nasal minute ventilation (MVₙ), a calibration constant (kₚ) may be applied to the nasal minute ventilation (MVₙ) such that the peak inspiratory flow (ûₚₑₐₖ) is approximately kₚMVₙ. The calibration constant (kₚ) can be between 3-6, or 3, 4, 5, or 6.

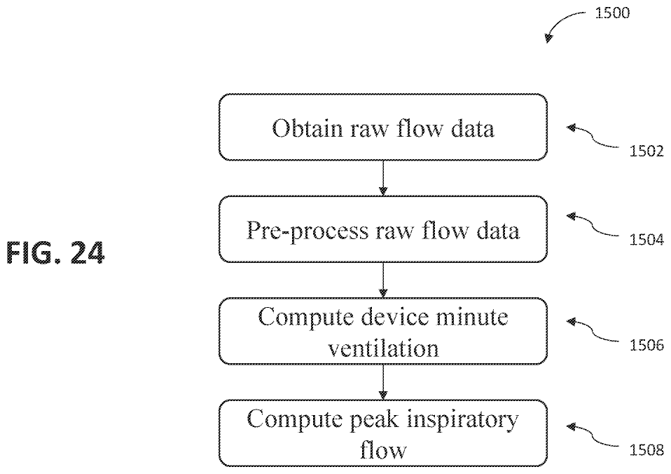

FIG. 24 illustrates a flowchart of a method of estimating peak inspiratory flow 1500. The method 1500 starts by obtaining raw flow data at step 1502. The raw flow data can be acquired from a flow rate sensor such as an ultrasonic flow sensor. The method 1500 can include pre-processing the raw flow data to remove unwanted signal components at step 1504. Removal of unwanted signal components is described in more detail above (e.g., step 1004 of method 1000, step 1104 of method 1100, step 1206 of method 1200, and step 1306 of method 1300). The unwanted signal components can be present from the flow generator motor. Once pre-processed, the flow data can be representative of patient breathing data. The method 1500 can include step 1506 where, assuming the data is of sufficient quality, the pre-processed data can be passed to the device minute ventilation algorithm to calculate the device minute ventilation (MV_{device}). Once the device minute ventilation (MV_{device}) is obtained, the method 1500 can optionally include converting the device minute ventilation (MV_{device}) into the nasal minute ventilation (MVₙ). As was described in more detail above, the nasal minute ventilation (MVₙ) can be estimated by converting and/or transforming the device minute ventilation (MV_{device}) using a scalar calibration constant. As was previously discussed, the scalar calibration constant can be determined using manually input parameters for the nasal cannula and current flow rate. In some configurations, if the nasal minute ventilation (MVₙ) is obtained, the peak inspiratory flow (ûₚₑₐₖ) can then be estimated by converting and/or transforming the nasal minute ventilation (MVₙ) using the scalar calibration constant (kₚ) at step 1508. As discussed above, the calibration constant (kₚ) can be between 3-6, or 3, 4, 5, or 6.

Estimating the peak inspiratory flow (ûₚₑₐₖ) can be useful as it can be used to provide a user or health care professional with a prompt to increase the flow rate setting if the controller determines that the patients' inspiratory demand is not being met. In some configurations, this can indicate to the user or healthcare professional that the patient may be entraining ambient air. This is undesirable as it can result in the dilution of the gas mixture provided, lowering the fraction of inspired oxygen (FiO₂) and potentially compromising some of the benefits of high flow therapy. In some examples, if a patient's peak inspiratory flow (ûₚₑₐₖ) is being met, this indicates to the user or health care professional that the O₂ fraction of the gases mixture output by the therapy device is (closely approximate) to the fraction of inspired oxygen (FiO₂) of the gases mixture inhaled by the patient. More details regarding the fraction of inspired oxygen (FiO₂) is discussed below. In some examples, a recommendation for flow rate can be displayed for the user or health care professional.

The trend and/or rate of change of the peak inspiratory flow (ûₚₑₐₖ) can provide the user and/or health care professional with valuable information. For example, if the peak inspiratory flow (ûₚₑₐₖ) increases over time, this can be an indication to the user and/or health care professional that the therapy parameters are effective as the patient's lungs are strengthening over time. However, if the peak inspiratory flow (ûₚₑₐₖ) decreases over time, this can indicate to the user that the patient's lung function is deteriorating. For example, a decrease in the peak inspiratory flow (ûₚₑₐₖ) can indicate worsening asthma symptoms, an increase in scar tissue, reduced patient energy, increased bacterial or viral infection, and increased fluid in the lungs. The trend information of the peak inspiratory flow (ûₚₑₐₖ) can be particularly useful when the nasal high flow system is used in a home setting. The trend information can help identify patient deterioration without the need for user know-how in interpreting individual data points and values.

Frequently, users and/or health care professionals will adjust oxygen concentration at the supply with the goal of increasing the fraction of inspired oxygen (FiO₂) instead of adjusting flow rate. While this can have the desired effect, it can be more beneficial to increase the flow rate instead. The fraction of inspired oxygen (FiO₂) setting at the point of supply does not necessarily coincide with the fraction of inspired oxygen (FiO₂) the patient actually receives because of interface leakage in an unsealed system. By instead increasing the device flow rate, this can help to bring the received fraction of inspired oxygen (FiO₂) closer towards the desired fraction of inspired oxygen (FiO₂) set at the point of supply. In some configurations, by understanding whether the device output is meeting patient peak inspiratory demand, flow rate can be adjusted accordingly.

The device controller can be configured to present audio and/or visual alarms or notifications for the user and/or health care professional if the peak inspiratory flow (ûₚₑₐₖ) or its rate of change is outside of preset thresholds. The thresholds can be suggested by the device or manually input by the user and/or healthcare professional. The peak inspiratory flow (ûₚₑₐₖ) can include a disconnection alarm when the peak inspiratory flow (Ûpeak) falls below a certain level. For example, if the peak inspiratory flow (ûₚₑₐₖ) drops below 5 L/min, it can illustrate a massive deterioration of the patient or a disconnection of the patient from the device.

The trend in the peak inspiratory flow (ûₚₑₐₖ) may also be displayed on a device screen. The device screen can provide a visualization of the peak inspiratory flow (Ûpeak) as a plot, chart, or graph. The controller can trigger notifications or alarms based on the peak inspiratory flow (ûₚₑₐₖ). The peak inspiratory flow may be transmitted to a mobile device or a remote server or a remote patient management system. The peak inspiratory flow may be incorporated into a patient report at the remote patient management system. The peak inspiratory flow and/or nasal minute ventilation may be transmitted to a remote patient management system at the end of a therapy session. Alternatively, this determined data may be transmitted at regular intervals to the remote system during therapy. Estimated tidal volume may also be transmitted to the remote patient management system. The nasal minute ventilation, peak inspiratory flow are also preferably displayed on a display of the respiratory therapy apparatus.

In some configurations the respiratory therapy apparatus may be controlled based on one of the determined patient parameters e.g. to achieve a set flow rate that matches or slightly exceeds the patient's peak inspiratory flow rate. The peak inspiratory flow of the patient may be estimated and compared to a set flow rate. The controller may increase the flow rate if the estimated peak inspiratory flow is above the set flow rate. If the flow rate is significantly above the estimate peak inspiratory flow of the patient, the controller may decrease the flow rate. Similarly, the controller may control the blower to provide a flow rate according to set threshold(s) and the estimated nasal minute ventilation of the user. A clinician may be able to manually set a nasal minute ventilation threshold. Then, if the controller estimates the current nasal minute ventilation over a period of time and finds it below a threshold, the flow rate may be adjusted (increased). The magnitude of adjustment may be based at least in part on a difference between the set threshold value and the estimated nasal minute ventilation value.

### Terminology

Unless the context clearly requires otherwise, throughout the description and the claims, the words “comprise,” “comprising,” and the like, are to be construed in an inclusive sense as opposed to an exclusive or exhaustive sense, that is to say, in the sense of “including, but not limited to.”

Although this disclosure has been described in the context of certain embodiments and examples, it will be understood by those skilled in the art that the disclosure extends beyond the specifically disclosed embodiments to other alternative embodiments and/or uses and obvious modifications and equivalents thereof. In addition, while several variations of the embodiments of the disclosure have been shown and described in detail, other modifications, which are within the scope of this disclosure, will be readily apparent to those of skill in the art. It is also contemplated that various combinations or sub-combinations of the specific features and aspects of the embodiments may be made and still fall within the scope of the disclosure. For example, features described above in connection with one embodiment can be used with a different embodiment described herein and the combination still fall within the scope of the disclosure. It should be understood that various features and aspects of the disclosed embodiments can be combined with, or substituted for, one another in order to form varying modes of the embodiments of the disclosure. Thus, it is intended that the scope of the disclosure herein should not be limited by the particular embodiments described above. Accordingly, unless otherwise stated, or unless clearly incompatible, each embodiment of this invention may comprise, additional to its essential features described herein, one or more features as described herein from each other embodiment of the invention disclosed herein.

Features, materials, characteristics, or groups described in conjunction with a particular aspect, embodiment, or example are to be understood to be applicable to any other aspect, embodiment or example described in this section or elsewhere in this specification unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing embodiments. The protection extends to any novel one, or any novel combination, of the features disclosed in this specification (including any accompanying claims, abstract and drawings), or to any novel one, or any novel combination, of the steps of any method or process so disclosed.

Furthermore, certain features that are described in this disclosure in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations, one or more features from a claimed combination can, in some cases, be excised from the combination, and the combination may be claimed as a subcombination or variation of a subcombination.

Moreover, while operations may be depicted in the drawings or described in the specification in a particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Further, the operations may be rearranged or reordered in other implementations. Those skilled in the art will appreciate that in some embodiments, the actual steps taken in the processes illustrated and/or disclosed may differ from those shown in the figures. Depending on the embodiment, certain of the steps described above may be removed, others may be added. Furthermore, the features and attributes of the specific embodiments disclosed above may be combined in different ways to form additional embodiments, all of which fall within the scope of the present disclosure. Also, the separation of various system components in the implementations described above should not be understood as requiring such separation in all implementations, and it should be understood that the described components and systems can generally be integrated together in a single product or packaged into multiple products.

For purposes of this disclosure, certain aspects, advantages, and novel features are described herein. Not necessarily all such advantages may be achieved in accordance with any particular embodiment. Thus, for example, those skilled in the art will recognize that the disclosure may be embodied or carried out in a manner that achieves one advantage or a group of advantages as taught herein without necessarily achieving other advantages as may be taught or suggested herein.

Conditional language, such as “can,” “could,” “might,” or “may,” unless specifically stated otherwise, or otherwise understood within the context as used, is generally intended to convey that certain embodiments include, while other embodiments do not include, certain features, elements, and/or steps. Thus, such conditional language is not generally intended to imply that features, elements, and/or steps are in any way required for one or more embodiments or that one or more embodiments necessarily include logic for deciding, with or without user input or prompting, whether these features, elements, and/or steps are included or are to be performed in any particular embodiment.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately,” “about,” “generally,” and “substantially” may refer to an amount that is within less than 10% of, within less than 5% of, within less than 1% of, within less than 0.1% of, and within less than 0.01% of the stated amount.

### Examples and/or Preferred Features

1. A respiratory device configured to deliver a respiratory therapy to a patient using an unsealed respiratory interface, the device comprising:
a controller, wherein the controller is configured to:
receive data of a parameter of a flow of gases of the respiratory device while the device is in use with an unsealed user interface, the parameter indicative of the patient's respiration,
determine a device minute ventilation or a parameter indicative of device minute ventilation, and
provide an indication of minute ventilation of a user.

2. The respiratory device of paragraph 1, wherein the controller is further configured to process the data of the parameter of the flow of gases to remove noise.

3. The respiratory device of paragraph 2, wherein the controller is configured to remove noise relating to the effect of a motor on the parameter of the flow of gases.

4. The respiratory device of paragraph 3, wherein the controller is configured to receive data regarding a motor speed, and the parameter of the flow of gases is discarded if the motor speed is below a pre-set threshold.

5. The respiratory device according to any one of paragraphs 1-3, wherein the controller is configured to discard the parameter of the flow of gases if the controller determines the parameter of the flow of gases is of insufficient quality.

6. The respiratory device of paragraph 5, wherein the parameter of the flow of gases is of insufficient quality because it includes large transient peaks.

7. The respiratory device according to any one of paragraphs 1-6, wherein determining a device minute ventilation comprises fitting a plurality of splines to the data of the parameter of the flow of gases, wherein the plurality of splines are fit using the least squares criterion and the device minute ventilation is determined by integrating along the plurality of splines.

8. The respiratory device according to any one of paragraphs 1-6, wherein determining a device minute ventilation comprises determining the integral of the absolute value of the first term of a line fitted to the data of the parameter of the flow of gases.

9 The respiratory device according to any one of paragraphs 1-6, wherein determining a device minute ventilation comprises determining the integral of the absolute value of a line fitted to the data of the parameter of the flow of gases, divided by a time range.

10. The respiratory device according to any one of paragraphs 1-6, wherein determining a device minute ventilation comprises an average of absolute values of a line fitted to the data of the parameter of the flow of gases across a range of time-points within a time range.

11. The respiratory device according to any one of paragraphs 1-10, wherein the controller is configured to compute a normalized device minute ventilation based on the device minute ventilation.

12. The respiratory device according to paragraph 11, wherein the controller is configured to calculate a noise correction factor correlated to the normalized device minute ventilation.

13. The respiratory device according to paragraphs 12, wherein the controller is configured to calculate a corrected device minute ventilation by relating the normalized device minute ventilation and the noise correction factor with the device minute ventilation.

14. The respiratory device according to any one of paragraphs 1-13, wherein the device minute ventilation is converted to a nasal minute ventilation.

15. The respiratory device of paragraph 14, wherein the device minute ventilation is converted to a nasal minute ventilation using a scalar calibration constant.

16. The respiratory device of paragraph 15, wherein the scalar calibration constant is determined by inputting patient interface parameters related to the nasal cannula or current flow rate.

17. The respiratory device of paragraph 15, wherein the scalar calibration constant is determined by inputting at least one of the cannula type, patient size, and naris diameter and/or amount of occlusion of the nares of the user.

18. The respiratory device of paragraph 15, wherein the scalar calibration constant is calculated by temporarily placing a sealed face mask over a patient's face while the patient is wearing an unsealed nasal cannula to measure at least one flow parameter of the respiratory device.

19. The respiratory device according to any one of paragraphs 1-18, wherein the controller is further configured to monitor at least one of the nasal minute ventilation, nasal minute ventilation rate of change, and nasal minute ventilation trends.

20. The respiratory device according to any one of paragraphs 1-19, wherein the respiratory device further comprises a display, wherein the display is configured to display data relating to the nasal minute ventilation.

21. The respiratory device according to any one of paragraphs 1-19, wherein the respiratory device further comprises a display, wherein the display is configured to display data relating to the nasal minute ventilation rate of change.

22. The respiratory device according to any one of paragraphs 1-19, wherein the respiratory device further comprises a display, wherein the display is configured to display data relating to the nasal minute ventilation trends.

23. The respiratory device according to any one of paragraphs 1-22, wherein the respiratory device is configured to trigger an alarm or notification when the nasal minute ventilation exceeds or falls below a preset threshold.

24. The respiratory device according to any one of paragraphs 1-22, wherein the respiratory device is configured to trigger an alarm or notification when the nasal minute ventilation rate exceeds or falls below a preset threshold.

25. The respiratory device according to any one of paragraphs 1-22, wherein the respiratory device is configured to trigger an alarm or notification when the nasal minute ventilation trends exceeds or falls below a preset threshold.

26. The respiratory device according to any one of paragraphs 1-25, wherein the respiratory device comprises a patient interface, wherein the patient interface comprises a nasal cannula.

27. The respiratory device according to any one of paragraphs 1-26, wherein the respiratory device is configured to deliver a nasal high flow therapy.

28. The respiratory device according to any one of paragraphs 1-27 further comprising a humidifier configured to humidify the gases flow to the patient.

29. A respiratory device configured to deliver a respiratory therapy to a patient using an unsealed respiratory interface, the device configured to provide information related to the patient's breathing, the device comprising:
a controller, wherein the controller is configured to:
receive data of a parameter of a flow of gases of the respiratory device, the parameter indicative of the patient's respiration,
process the data of the parameter of the flow of gases to remove noise,
determine whether the parameter of the flow of gases is of sufficient quality,
determine a device minute ventilation, and
convert the device minute ventilation to a nasal minute ventilation using a scalar calibration constant.

30. The respiratory device of paragraph 29, wherein the controller is configured to remove noise relating to the effect of a motor on the parameter of the flow of gases.

31. The respiratory device of paragraph 30, wherein the controller is configured to receive data regarding a motor speed, and the parameter of the flow of gases is discarded if the motor speed is below a pre-set threshold.

32. The respiratory device of paragraph 31, wherein the parameter of the flow of gases is of insufficient quality because it includes large transient peaks.

33. The respiratory device according to any one of paragraphs 29-32, wherein determining a device minute ventilation comprises fitting a plurality of splines to the data of the parameter of the flow of gases, wherein the plurality of splines are fit using the least squares criterion and the device minute ventilation is determined by integrating along the plurality of splines.

34. The respiratory device according to any one of paragraphs 29-32, wherein determining a device minute ventilation comprises determining the integral of the absolute value of the first term of a line fitted to the data of the parameter of the flow of gases.

35. The respiratory device according to any one of paragraphs 29-32, wherein determining a device minute ventilation comprises the integral of the absolute value of a line fitted to the data of the parameter of the flow of gases, divided by a time range.

36. The respiratory device according to any one of paragraphs 29-32, wherein determining a device minute ventilation comprises an average of absolute values of a line fitted to the data of the parameter of the flow of gases across a range of time-points within a time range.

37. The respiratory device according to any one of paragraphs 29-36, wherein the controller is configured to compute a normalized device minute ventilation based on the device minute ventilation.

38. The respiratory device according to paragraph 37, wherein the controller is configured to calculate a noise correction factor correlated to the normalized device minute ventilation.

39. The respiratory device according to paragraphs 38, wherein the controller is configured to calculate a corrected device minute ventilation by relating the normalized device minute ventilation and the noise correction factor with the device minute ventilation.

40. The respiratory device of paragraph 39, wherein the scalar calibration constant is determined by inputting patient interface parameters related to the nasal cannula or current flow rate.

41. The respiratory device of paragraph 39, wherein the scalar calibration constant is determined by inputting at least one of the cannula type, patient size, and naris diameter.

42. The respiratory device of paragraph 39, wherein the scalar calibration constant is calculated by temporarily placing a sealed face mask over a patient's face while the patient is wearing a nasal cannula to measure at least one flow parameter of the respiratory device.

43. The respiratory device according to any one of paragraphs 29-42, wherein the controller is further configured to monitor at least one of the nasal minute ventilation, nasal minute ventilation rate of change, and nasal minute ventilation trends.

44. The respiratory device according to any one of paragraphs 29-43, wherein the respiratory device further comprises a display, wherein the display is configured to display data relating to the nasal minute ventilation.

45. The respiratory device according to any one of paragraphs 29-43, wherein the respiratory device further comprises a display, wherein the display is configured to display data relating to the nasal minute ventilation rate of change.

46. The respiratory device according to any one of paragraphs 29-43, wherein the respiratory device further comprises a display, wherein the display is configured to display data relating to the nasal minute ventilation trends.

47. The respiratory device according to any one of paragraphs 26-46, wherein the respiratory device is configured to trigger an alarm or notification when the nasal minute ventilation exceeds or falls below a preset threshold.

48. The respiratory device according to any one of paragraphs 29-46, wherein the respiratory device is configured to trigger an alarm or notification when the nasal minute ventilation rate of change exceeds or falls below a preset threshold.

49. The respiratory device according to any one of paragraphs 29-46, wherein the respiratory device is configured to trigger an alarm or notification when the nasal minute ventilation trends exceeds or falls below a preset threshold.

50. The respiratory device according to any one of paragraphs 29-49, wherein the respiratory device comprises a patient interface, wherein the patient interface comprises a nasal cannula.

51. The respiratory device according to any one of paragraphs 29-50, wherein the respiratory device is configured to deliver a nasal high flow therapy.

52. The respiratory device according to any one of paragraphs 29-51 further comprising a humidifier configured to humidify the gases flow to the patient.

53. A respiratory device configured to deliver a respiratory therapy to a patient using an unsealed respiratory interface, the device configured to provide information related to the patient's breathing, the device comprising:
a controller, wherein the controller is configured to:
   receive data of a parameter of a flow of gases of the respiratory device, the parameter indicative of the patient's respiration, and
   determine a patient peak inspiratory flow.

54. The respiratory device of paragraph 53, wherein the controller is configured to determine a device minute ventilation.

55. The respiratory device of paragraph 54, wherein the patient peak inspiratory flow is based on the device minute ventilation.

56. The respiratory device of any one of paragraphs 53-55, wherein the controller is further configured to process the data of the parameter of the flow of gases to remove noise.

57. The respiratory device of paragraph 56, wherein the controller is configured to remove noise relating to the effect of a motor on the parameter of the flow of gases.

58 The respiratory device of paragraph 57, wherein the controller is configured to receive data regarding a motor speed, and the parameter of the flow of gases is discarded if the motor speed is below a pre-set threshold.

59. The respiratory device according to any one of paragraphs 53-58, wherein the controller is configured to discard the parameter of the flow of gases if the controller determines the parameter of the flow of gases is of insufficient quality.

60. The respiratory device of paragraph 59, wherein the parameter of the flow of gases is of insufficient quality because it includes large transient peaks.

61. The respiratory device according to any one of paragraphs 54-60, wherein determining the device minute ventilation comprises fitting a plurality of splines to the data of the parameter of the flow of gases, wherein the plurality of splines are fit using the least squares criterion and the device minute ventilation is determined by integrating along the plurality of splines.

62. The respiratory device according to any one of paragraphs 54-61, wherein determining the device minute ventilation comprises determining the integral of the absolute value of the first term of a line fitted to the data of the parameter of the flow of gases.

63. The respiratory device according to any one of paragraphs 54-62, wherein determining the device minute ventilation comprises the integral of the absolute value of a line fitted to the data of the parameter of the flow of gases, divided by a time range.

64. The respiratory device according to any one of paragraphs 54-63, wherein determining the device minute ventilation comprises an average of absolute values of a line fitted to the data of the parameter of the flow of gases across a range of time-points within a time range.

65. The respiratory device according to any one of paragraphs 53-64, wherein the controller is configured to compute a normalized device minute ventilation based on the device minute ventilation.

66. The respiratory device according to paragraph 65, wherein the controller is configured to calculate a noise correction factor correlated to the normalized device minute ventilation.

67. The respiratory device according to paragraphs 66, wherein the controller is configured to calculate a corrected device minute ventilation by relating the normalized device minute ventilation and the noise correction factor with the device minute ventilation.

68. The respiratory device according to any one of paragraphs 54-67, wherein the controller is further configured to convert the device minute ventilation to a nasal minute ventilation

69. The respiratory device of paragraph 68, wherein the device minute ventilation is converted to a nasal minute ventilation using a scalar calibration constant.

70. The respiratory device of paragraph 69, wherein the scalar calibration constant is determined by inputting patient interface parameters related to the nasal cannula or current flow rate.

71. The respiratory device of paragraph 70, wherein the scalar calibration constant is determined by inputting at least one of the cannula type, patient size, and naris diameter.

72. The respiratory device of paragraph 71, wherein the scalar calibration constant is calculated by temporarily placing a sealed face mask over a patient's face while the patient is wearing a nasal cannula to measure at least one flow parameter of the respiratory device.

73. The respiratory device according to any one of paragraphs 68-72, wherein the patient peak inspiratory flow is determined by converting the nasal minute ventilation by applying a calibration constant.

74. The respiratory device of paragraph 73, wherein the calibration constant is 3.

75. The respiratory device of paragraph 73, wherein the calibration constant is 4.

76. The respiratory device of paragraph 73, wherein the calibration constant is 5.

77. The respiratory device of paragraph 73, wherein the calibration constant is 6.

78. The respiratory device of paragraph 73, wherein the calibration constant is between 3 and 6.

79. The respiratory device according to any one of paragraphs 53-78 wherein the controller is further configured to monitor at least the peak inspiratory flow or the rate of change of the peak inspiratory flow.

80. The respiratory according to any one of paragraphs 53-79, wherein the respiratory device further comprises a display, wherein the display is configured to display data relating to the peak inspiratory flow.

81. The respiratory device according to any one of paragraphs 53-80, wherein the respiratory device further comprises a display, wherein the display is configured to display data relating to the rate of change of the peak inspiratory flow.

82. The respiratory device according to any one of paragraphs 53-81, wherein the respiratory device is configured to trigger an alarm or notification when the peak inspiratory flow exceeds or falls below a preset threshold.

83. The respiratory device according to any one of paragraphs 53-82, wherein the respiratory device is configured to trigger an alarm or notification when the rate of change of the peak inspiratory flow exceeds or falls below a preset threshold.

84. The respiratory device according to any one of paragraphs 53-83, wherein the respiratory device comprises a patient interface, wherein the patient interface comprises a nasal cannula.

85. The respiratory device according to any one of paragraphs 53-84, wherein the respiratory device is configured to deliver a nasal high flow therapy.

86. The respiratory device according to any one of paragraphs 54-85 further comprising a humidifier configured to humidify the gases flow to the patient.

87. A respiratory device configured to deliver a respiratory therapy to a patient using an unsealed respiratory interface, the device configured to provide information related to the patient's breathing, the device comprising:
a controller, wherein the controller is configured to:
   receive a flow rate of a flow of gases of the respiratory device while the device is in use with an unsealed user interface,
   determine a respiration parameter based on the received flow rate of gases, wherein the respiratory parameter is indicative of patient minute ventilation.

88. A respiratory device of paragraph 87 wherein the controller is configured to display the respiration parameter on a graphical user interface.

89. A respiratory device according to any one of paragraphs 87-88 wherein the controller is configured to determine a device minute ventilation based on the flow of the gases, and the controller configured to determine the patient minute ventilation based on the device minute ventilation.

90. A respiratory device according to any one of paragraphs 87-89 wherein the controller is further configured to process the flow rate of the flow of gases to remove noise.

91. The respiratory device of paragraph 90, wherein the controller is configured to remove noise relating to the effect of a motor on the parameter of the flow of gases.

92. The respiratory device of paragraph 91, wherein the controller is configured to receive data regarding a motor speed, and the parameter of the flow of gases is discarded if the motor speed is below a pre-set threshold.

93. The respiratory device according to any one of paragraphs 87-92, wherein the controller is configured to discard the flow rate of the flow of gases if the controller determines the flow rate of the flow of gases is of insufficient quality.

94. The respiratory device of paragraph 93, wherein the flow rate of the flow of gases is of insufficient quality because it includes large transient peaks.

95. A respiratory device according to any one of paragraphs 87-94, wherein the controller is configured to determine the device minute ventilation according to any one of paragraphs 7 to 10, and patient minute ventilation may be calculated as defined in any one or more of the above paragraphs.

96. A respiratory device configured to deliver a respiratory therapy to a patient using an unsealed respiratory interface, the device configured to provide information related to the patient's breathing, the device comprising:
a controller, wherein the controller is configured to:
   receive a flow rate of a flow of gases of the respiratory device while the device is in use with an unsealed user interface,
   determine a patient peak inspiratory flow based on the received flow rate of gases.

97. The respiratory device of paragraph 96, wherein the controller is configured to display the patient peak inspiratory flow on a graphical user interface.

98. The respiratory device according to any one of paragraphs 96-97, wherein the controller is configured to determine the patient peak inspiratory flow based on a device minute ventilation.

99. The respiratory device according to any one of paragraphs 96-98, wherein the device minute ventilation is based on the flow rate of the flow of gases, wherein the flow rate of the flow of gases is pre-processed according to any one of paragraphs 56-60.

100. The respiratory device according to any one of paragraphs 96-99, wherein the device minute ventilation is calculated according to any one of paragraphs 61-64.

101. The respiratory device according to any one of paragraphs 96-100, wherein the controller is configured to convert the device minute ventilation to a nasal minute ventilation according to any of paragraphs 68-78.

102. The respiratory device according to any one of paragraphs 96-101, wherein the controller is configured to monitor at least the peak inspiratory flow or the rate of change of the peak inspiratory flow.

103. The respiratory device according to any one of paragraphs 96-102, wherein the respiratory device further comprises a display, the display is configured to display data relating to at least one of a peak inspiratory flow or a rate of change of the peak inspiratory flow.

104. The respiratory device according to any one of paragraphs 96-103, wherein the respiratory device is configured to trigger an alarm or notification when the peak inspiratory flow exceeds or falls below a preset threshold.

105. The respiratory device according to any one of paragraphs 96-104, wherein the respiratory device is configured to trigger an alarm or notification when the rate of change of the peak inspiratory flow exceeds or falls below a preset threshold.

106. The respiratory device according to any one of paragraphs 1-28, wherein the controller is configured to determine a tidal volume and trigger an alarm or notification when the tidal volume falls below a preset threshold.

107. The respiratory device according to any one of paragraphs 29-52, wherein the controller is configured to determine a tidal volume and trigger an alarm or notification when the tidal volume falls below a preset threshold.

108. The respiratory device according to any one of paragraphs 53-86 wherein the controller is configured to determine a tidal volume and trigger an alarm or notification when the tidal volume falls below a preset threshold.

109. The respiratory device according to any one of paragraphs 87-95, wherein the controller is configured to determine a tidal volume and trigger an alarm or notification when the tidal volume falls below a preset threshold.

110. The respiratory device according to any one of paragraphs 96-105, wherein the controller is configured to determine a tidal volume and trigger an alarm or notification when the tidal volume falls below a preset threshold.

111. The respiratory device according to any one of paragraphs 1-28, wherein the controller is configured to normalize a corrected device minute ventilation using a device flow rate.

112. The respiratory device according to any one of paragraphs 111, wherein the respiratory device further comprises a display configured to display data relating to at least one of the corrected device minute ventilation, corrected device minute ventilation rate of change, and corrected device minute ventilation trends.

113. The respiratory device according to any one of paragraphs 111-112, wherein the respiratory device is configured to trigger an alarm or notification when at least one of the corrected device minute ventilation exceeds or falls below a preset threshold, the corrected device minute ventilation rate of change exceeds or falls below a preset threshold, and the corrected device minute ventilation trends exceeds or falls below a preset threshold.

114. The respiratory device according to any one of paragraphs 29-52, wherein the controller is configured to normalize a corrected device minute ventilation using a device flow rate.

115. The respiratory device according to any one of paragraphs 114, wherein the respiratory device further comprises a display configured to display data relating to at least one of the corrected device minute ventilation, corrected device minute ventilation rate of change, and corrected device minute ventilation trends.

116. The respiratory device according to any one of paragraphs 114-115, wherein the respiratory device is configured to trigger an alarm or notification when at least one of the corrected device minute ventilation exceeds or falls below a preset threshold, the corrected device minute ventilation rate of change exceeds or falls below a preset threshold, and the corrected device minute ventilation trends exceeds or falls below a preset threshold.

117. The respiratory device according to any one of paragraphs 53-86 wherein the controller is configured to normalize a corrected device minute ventilation using a device flow rate.

118. The respiratory device according to paragraph 117, wherein the respiratory device further comprises a display configured to display data relating to at least one of the corrected device minute ventilation, corrected device minute ventilation rate of change, and corrected device minute ventilation trends.

119. The respiratory device according to any one of paragraphs 117-118, wherein the respiratory device is configured to trigger an alarm or notification when at least one of the corrected device minute ventilation exceeds or falls below a preset threshold, the corrected device minute ventilation rate of change exceeds or falls below a preset threshold, and the corrected device minute ventilation trends exceeds or falls below a preset threshold.

120. The respiratory device according to any one of paragraphs 87-95, wherein the controller is configured to normalize a corrected device minute ventilation using a device flow rate.

121. The respiratory device according to any one of paragraphs 120, wherein the respiratory device further comprises a display configured to display data relating to at least one of the corrected device minute ventilation, corrected device minute ventilation rate of change, and corrected device minute ventilation trends.

122. The respiratory device according to any one of paragraphs 120-121, wherein the respiratory device is configured to trigger an alarm or notification when at least one of the corrected device minute ventilation exceeds or falls below a preset threshold, the corrected device minute ventilation rate of change exceeds or falls below a preset threshold, and the corrected device minute ventilation trends exceeds or falls below a preset threshold.

123. The respiratory device according to any one of paragraphs 96-105, wherein the controller is configured to normalize a corrected device minute ventilation using a device flow rate.

124. The respiratory device according to any one of paragraphs 123, wherein the respiratory device further comprises a display configured to display data relating to at least one of the corrected device minute ventilation, corrected device minute ventilation rate of change, and corrected device minute ventilation trends.

125. The respiratory device according to any one of paragraphs 123-124, wherein the respiratory device is configured to trigger an alarm or notification when at least one of the corrected device minute ventilation exceeds or falls below a preset threshold, the corrected device minute ventilation rate of change exceeds or falls below a preset threshold, and the corrected device minute ventilation trends exceeds or falls below a preset threshold.

126. A respiratory device configured to deliver a respiratory therapy to a patient using an unsealed respiratory interface, the device comprising:
a blower
a humidifier
a housing to retain the blower and the humidifier being at least partially retained within the housing,
a gases inlet formed within the housing
a gases flow path extending from the gases inlet through the blower and the humidifier,
the humidifier in fluid communication with the blower,
the blower in fluid communication with the gases inlet,
one or more flow sensors located in the gases flow path, the one or more flow sensors configured to measure flow data of the gases
a controller located within the housing, the controller in electrical communication with the blower, the humidifier and the one or more flow sensors, the controller configured to control operation of the blower and the humidifier,
the controller further configured to:
   receive flow data from the one or more flow sensors, the controller configured to process the received flow data to filter the flow data to remove noise and determine a minute ventilation to user,
   provide the determined minute ventilation to a display,
a display in electrical communication with the display, the minute ventilation to the user is presented on the display.

127. The respiratory device according to paragraph 126 wherein the controller is configured to perform the functions according to any one or more of paragraphs 1-125.

128. A respiratory system for managing a user comprising:
a respiratory device according to any one of paragraphs 1 to 127
a remote patient management system in wireless communication with the respiratory device, the remote patient management system is configured receive the minute ventilation of a user and store the received minute ventilation and associate the stored minute ventilation with a specific user.

1. A respiratory device configured to deliver a respiratory therapy to a patient using an unsealed respiratory interface, the device comprising:
a controller, wherein the controller is configured to:
receive data of a parameter of a flow of gases of the respiratory device while the device is in use with an unsealed user interface, the parameter indicative of the patient's respiration,
determine a device minute ventilation or a parameter indicative of device minute ventilation, and
provide an indication of minute ventilation of a user.

2-6. (canceled)

7. The respiratory device of claim 1, wherein determining a device minute ventilation comprises fitting a plurality of splines to the data of the parameter of the flow of gases, wherein the plurality of splines are fit using the least squares criterion and the device minute ventilation is determined by integrating along the plurality of splines.

8. The respiratory device of claim 1, wherein determining a device minute ventilation comprises determining the integral of the absolute value of the first term of a line fitted to the data of the parameter of the flow of gases.

9. The respiratory device of claim 1, wherein determining a device minute ventilation comprises determining the integral of the absolute value of a line fitted to the data of the parameter of the flow of gases, divided by a time range.

10. The respiratory device of claim 1, wherein determining a device minute ventilation comprises an average of absolute values of a line fitted to the data of the parameter of the flow of gases across a range of time-points within a time range.

11-13. (canceled)

14. The respiratory device of claim 1, wherein the device minute ventilation is converted to a nasal minute ventilation.

15. The respiratory device according to claim 14, wherein the device minute ventilation is converted to a nasal minute ventilation using a scalar calibration constant.

16. The respiratory device of claim 1, wherein the controller is further configured to monitor at least one of the nasal minute ventilation, nasal minute ventilation rate of change, and nasal minute ventilation trends.

17. The respiratory device of claim 1, wherein the respiratory device further comprises a display, wherein the display is configured to display data relating to the nasal minute ventilation.

18. The respiratory device of claim 1, wherein the respiratory device further comprises a display, wherein the display is configured to display data relating to the nasal minute ventilation rate of change.

19. The respiratory device of claim 1, wherein the respiratory device further comprises a display, wherein the display is configured to display data relating to the nasal minute ventilation trends.

20. The respiratory device of claim 1, wherein the respiratory device is configured to trigger an alarm or notification when the nasal minute ventilation exceeds or falls below a preset threshold.

21. The respiratory device of claim 1, wherein the respiratory device is configured to trigger an alarm or notification when the nasal minute ventilation rate exceeds or falls below a preset threshold.

22. The respiratory device of claim 1, wherein the respiratory device is configured to trigger an alarm or notification when the nasal minute ventilation trends exceeds or falls below a preset threshold.

23. The respiratory device of claim 1, wherein the respiratory device comprises a patient interface, wherein the patient interface comprises a nasal cannula.

24. The respiratory device of claim 1, wherein the respiratory device is configured to deliver a nasal high flow therapy.

25. The respiratory device of claim 1, further comprising a humidifier configured to humidify the gases flow to the patient.

26-67. (canceled)

68. The respiratory device of claim 15, wherein the scalar calibration constant is determined by inputting patient interface parameters related to the unsealed respiratory interface or current flow rate of the flow of gases.

69. The respiratory device of claim 15, the scalar calibration constant is determined by inputting at least one of the unsealed respiratory interface type, patient size, and naris diameter and/or amount of occlusion of the nares of the patient.

70. The respiratory device of claim 15, the scalar calibration constant is calculated by temporarily placing a sealed face mask over the patient's face while the patient is wearing the unsealed respiratory interface to measure at least one flow parameter of the respiratory device.
